(19) United States
(12) Patent Application Publication
(10) Pub. No.: US 2021/0038145 A1
(43) Pub. Date: Feb. 11, 2021

(54) POSITIONAL FEEDBACK DEVICES

(71) Applicant: Digihealth Pty Ltd, Bulleen (AU)

(72) Inventors: Michael Simonetti, Melbourne (AU); Brian Khuu, Melbourne (AU); Jian Du, Melbourne (AU); Xin He, Melbourne (AU); Elaine Wong, Melbourne (AU)

(21) Appl. No.: 16/921,430

(22) Filed: Jul. 6, 2020

Related U.S. Application Data
(63) Continuation of application No. 15/513,821, filed on Mar. 23, 2017, now abandoned.
(60) Provisional application No. 62/065,034, filed on Oct. 17, 2014.

(30) Foreign Application Priority Data
Oct. 17, 2014 (AU) 2014250691
Mar. 27, 2015 (AU) 2015901109

(51) Int. Cl. A61B 5/00; G01B 11/16; G01B 7/16; A61B 5/11; G01D 5/165

(52) U.S. Cl. CPC A61B 5/4561; A61B 2562/0219; A61B 5/458; A61B 5/4595; A61B 5/4585; A61B 5/6823; A61B 5/4576; G01B 11/18; G01B 7/20; A61B 5/0004; A61B 5/002; A61B 5/11; A61B 5/486; A61B 5/6822; A61B 5/6824; A61B 5/6828; A61B 5/6829; G01D 5/165; A61B 5/4566; A61B 2562/043; A61B 2562/028; A61B 2562/0261; A61B 5/459; A61B 5/1116; A61B 2505/09; A61B 5/68335

(57) ABSTRACT

An apparatus comprising at least one sensor to detect the position and/or orientation of a body portion of a subject, the sensor in communication with a computing device to process sensor data and optionally a transmitter to transmit sensor data between the sensor and the computing device and/or one or more computing devices.

### BACKGROUND OF THE INVENTION

There is an ongoing need to sense the position and/or orientation of body parts for humans and animals. For example, in relation to sensing posture—more physical injury is caused by people placing themselves in bad posture over extended periods than from sharp forces on their body. This is because bad posture over a period of time applies more force over time, compared to forces of a similar magnitude applied as a short impulse. The kinds of posture that could result in long term posture related injuries also include posture whilst using a computer, lifting heavy objects, or back strain whilst undertaking a repetitive physical task, such as hammering nails in an awkward position.

Currently there are a number of methods attempting to curb such posture issues. For example, there is a direct message advertising campaign that carries a message on how to maintain good posture in the workplace and home while using computers. There is also training given to employees who often have to carry heavy items, especially with common tasks such as restocking high shelves.

Bio Corrective Physiotherapist use posture tapes to fix bad posture after it happens. This often requires trips to the Physiotherapist, which takes up their time that could be better spent on less preventable injuries and adds to the overall expense of healthcare to society.

In the medical field there are already expensive devices (often optical fibres), used to obtain, for example an exact angle of the body for research purpose. This is important, but since it is for research and is bulky and expensive, it is unsuitable for widespread prevention of back injuries over time.

There are also simple programs that at specific intervals recommend certain stretch exercises to relieve muscle strain before using a computer again. In addition, there are devices such as the LUMOBack that measures how often you are sitting down or running, or conducting other activities based on a single orientation sensor attached to the lower back. Often they encourage a healthier lifestyle by turning good activities like running into a score, which essentially gamifies and provide incentives to do better and be healthier.

None of the current methods sufficiently incentivise the user to correct their behaviour, or are priced at a point for general usage (and this thus limits the method's effectiveness to a reactionary solution, as opposed to a preventative solution).

In a similar way, there is an ongoing need for cost effective, generally available position and/or orientation detection devices to aid in developing sporting skills or other movement or posture based activities.

The reference to any prior art in this specification is not, and should not be taken as, an acknowledgement or any form of suggestion that the prior art forms part of the common general knowledge.

### SUMMARY OF THE INVENTION

According to one aspect of the invention, there is provided an apparatus comprising at least one sensor to detect the position and/or orientation of a body portion of a subject, the sensor in communication with a computing device to process sensor data and optionally a transmitter to transmit sensor data.

In another aspect, the invention provides a system for detecting the position and/or orientation of a body portion of a subject comprising a sensor and a computing device in communication with the sensor, the computing device able to process sensed data preferably into a form suitable for providing feedback.

The invention also provides a body portion position and/or orientation detection apparatus comprising: a sensor device configured to be attached to a user, the sensor device comprising: a flexible sensor and a microprocessor configured to receive and process data from the sensor about movement of the body portion.

In another aspect, the invention provides a method for detecting the position and/or orientation of a body portion comprising: receiving a plurality of resistance values from a flexible sensor strip in contact with the body portion and processing the resistance values to determine a body portion position and/or orientation wherein the processing step optionally comprises comparing one or more resistance values so as to arrive at a relative position and/or orientation of the body part.

The invention also provides a method of providing body portion position and/or orientation data comprising: receiving by a microprocessor data from a flexible sensor, the method comprising determining by the microprocessor a positional and/or orientation description of the body portion based on the received sensor data, wherein determining positional and/or orientation description based on the sensed data comprises: processing sensor input data and optionally triggering feedback based on the positional and/or orientation description of the body portion and wherein the processing step optionally comprises comparing one or more resistance values so as to arrive at a relative position and/or orientation of the body part.

In another aspect, the invention provides a computer program on a computer readable medium which when executed by a computer, is arranged to receive a plurality of resistance values from a flexible sensor strip in contact with a body portion and processing the resistance values to determine a body portion position and/or orientation wherein the processing step optionally comprises comparing one or more resistance values so as to arrive at a relative position and/or orientation of the body part.

In another aspect, the invention provides a non-transitory computer readable medium having stored thereupon computing instructions comprising: a code segment to receive by a microprocessor data from a body portion sensor; a code segment to process the received sensor data; a code segment to determine by the microprocessor a position and/or orientation description of the user based on the received sensor data; and optionally a code segment to trigger by the microprocessor feedback based on position and/or orientation of the body portion wherein the processing step optionally comprises comparing one or more resistance values so as to arrive at a relative position and/or orientation of the body part.

In a another aspect of the invention, there is provided an apparatus comprising at least one sensor to detect the position and/or orientation of a body portion of a subject, the sensor in communication with a computing device to process sensor data and optionally a transmitter to transmit sensor data between the sensor and the computing device and/or one or more computing devices.

Information such as that relating to position, orientation, posture and physical location can be sensed in relation to any suitable body portion. For example, it may be a joint or a series of joints such as the spine, a shoulder, an elbow, a wrist, a digital joint, a hip, a knee, an ankle, or it may be a particular location such as particular bony prominence or other anatomical landmark. The invention is useful for health and medical uses such as to fix postural or repetitive strain type injuries. It may equally be used for other applications, such as in sport or other human movement areas. In some embodiments, the invention is used for example to monitor and alter a particular human movement, such as a golf swing, a tennis swing, a football kicking action etc.

In some embodiments without the optional transmitter, the computing device is in physical communication with the sensor. In some embodiments, the computing device is not physically near the sensor so that communication must be via wireless or by another means. Sensor data may be processed for example by components of the sensor prior to transmission or it may be communicated to the computing device and then processed.

In some preferred embodiments, the sensor comprises a disposable strip which can be altered to a required length and the strip optionally comprises one or more predetermined locations at which length can be reduced. As an example, the strip may comprise one or more perforations at one or more predetermined locations so that it may be torn or cut at a certain length to suit the length of the body part of a particular subject (for example, the vertebral column, an arm, etc). In such examples, one end of the strip may for example plug directly into a device such as a data handler, a computing device and/or a transmitter whilst the other end comprises one or more of such locations at which length can be altered.

Alterations in size and or shape of a strip according to the invention may be attained by any suitable means. In some embodiments, the strip is telescopic so that the strip may be slid down to an appropriate size. In other embodiments, the strip comprises a series of separate smaller strips which can be engaged with one another to create a particular size and/or shape.

In some embodiments of the invention, the sensor comprises a resistive ink and a conductive ink. Each of these inks may be of any suitable type.

In some embodiments, the sensor comprises an adhesive section to adhere in working proximity to the body part of the subject wherein adhesion to the body optionally comprises one or more of adhesion to a close fitting garment and adhesion to the skin (and or hair) of the subject. The adhesive section may comprise any suitable adhesive. In some embodiments it is a medical grade adhesive suitable for direct contact with skin.

In some embodiments, the sensor comprises a perforated section to adhere to the body of the subject wherein adhesion to the body optionally comprises one or more of perforations to a close fitting garment and adhesion to the skin (and or hair) of the subject. The perforated section may comprise any suitable perforation. In some embodiments it is a manufacturing perforation suitable for direct contact with skin.

In some embodiments, the sensor comprises folds or manufacturing scoring sections to adhere to the body of the subject wherein adhesion to the body optionally comprises one or more of scores to a close fitting garment and adhesion to the skin (and or hair) of the subject. The folded sections may comprise any suitable folding. In some embodiments it is a manufactured scoring or folding suitable for direct contact with skin.

Some embodiments of the invention comprise one or more disposable components for low-cost replacement which optionally comprises a sensor strip. In some of these embodiments, the sensor strip itself is disposable.

Some embodiments of the invention further comprise a data handler to receive sensor data from the sensor and optionally store, and/or manage communication of said data to a computing device. Communication between the sensor and data handler may be by any suitable means, for example physical connection, wireless communication, Bluetooth, zigby, cellular network, computer network, satellite and so on.

In some embodiments, the sensor communicates directly with the computing device. Such communication may be by any suitable means, including physical connection, wireless communication, Bluetooth, zigby, cellular network, computer network, satellite and so on.

According to another aspect of the invention there is provided, a system for detecting the position and/or orientation of a body portion of a subject comprising a sensor and a computing device in communication with the sensor, the computing device able to process sensed data preferably into a form suitable for providing feedback. Some embodiments of the invention provide a system comprising a mobile computing device to notify the subject with feedback and optionally store and/or process sensed posture data.

In some important embodiments, the apparatus and/or system of the invention uses 3 key parts. First it uses a wearable, adhesive tape sensor which can accurately measure the entire spine, calibrating to both neutral and best-achievable spinal position for a user. An important aspect of this is the adhesive and folding aspects of the sensors which stick to the body and greatly reduce measurement errors. In these embodiments, the tape sensors connect to a separate processing and transmission device as the sensors are intended for single to a few uses only (they are disposable). In some embodiments, the transmission device connects to a smart mobile phone over bluetooth and allows gamification incentives, recording of the postural history of a user. With software upgrades to the transmission device and mobile, the disposable adhesive strip allows for future applications to different parts of the body, for example the shoulders, which are also important in terms of posture correction.

In some implementations of the invention, there is provided an apparatus or system comprising: a flexible sensor; an accelerometer; optionally a gyroscope; a data collection unit; a communication device (which is optionally Bluetooth); a smart phone; and a smart phone application (app) capable of processing resistance measurements to determine a position and/or orientation of a body portion.

In some embodiments of the invention, the sensor system comprises a plurality of pairs of sensors in a vertically cascading format.

Some embodiments of the apparatus or system of the invention comprise a scheduler to minimise stalling other functions while a routine is waiting for the next round to run again.

In some embodiments there is provided a method of sensing the position and/or orientation of a body portion comprising: attaching a sensor to or in close proximity to the body part, wherein the sensor comprises resistive and conductive ink and an adhesive strips; creating a communication-enabling connection between the sensor and a microprocessor transmission device; connecting the transmission device to a mobile phone using Bluetooth; sending raw data from the transmission device in real time for each sensor on the strip to the mobile phone; running on the mobile phone an application which is calibrated first at a neutral body portion position for the person; collating on the mobile phone the real-time resistive data; providing a visual demonstration of said data; alerting the user if the position and/or orientation meets or fails to meet pre-set criteria; and logging on the mobile phone the data and therefore history of the data provided by the strip via the transmission device.

Throughout this specification (including any claims which follow), unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps.

### BRIEF DESCRIPTION OF THE DRAWINGS

FIG. 1 depicts an example PCB design according to one embodiment of the invention.

FIG. 2 shows a breakdown of various subsystems of an example system according to the invention.

FIG. 3 shows measurement of an optical fibre based sensor. −90 is left, +90 is to the right.

FIG. 4 shows a typical foil strain gauge design.

FIG. 5 shows an example of interweaved elastic tape used for clothing

FIG. 6 shows resistance vs elongation from 40 mm relationship of Bare Conductive painted elastic sensor.

FIG. 7 shows resistance vs elongation from 40 mm relationship from one batch of Bare Conductive soaked elastics.

FIG. 8 shows variance of infused sensor stretch test.

FIG. 9 shows that as the sensors bend, micro cracks appear ad the resistance increases.

FIG. 10 shows resistance vs flex angle profile of two individual SpectraSymbol 2.2″ FS flex sensors. Both data series are normalised by subtracting out the intrinsic resistance. A 4th order polynomial trend line is included.

FIG. 11 shows SpectraSymbol 2″ FS flex sensor. Resistive element of the sensor is the black strip, with conducting elements placed on top to reduce resistance. Two sensors are arranged in opposite directions forming half of a Wheatstone bridge.

FIG. 12 shows normalised voltage of SS flex sensor pairs. A sweep left (+)90 and right (−)90 results in hysteresis

FIG. 13 shows a handmade resistive sensor using BC paint, exible PET card and aluminium foil.

FIG. 14 shows resistance vs flex angle profile of hand made resistive ink based flex sensors using Bare Conductive paint, aluminium foil on flexible PET card. Both data series are normalised by subtracting out the intrinsic resistance.

FIG. 15 shows printing pass 1—Carbon (cyan). Pass 2 & 3—Carbon and Silver (black)

FIG. 16 shows single and double pass resistance curing in open air.

FIG. 17 is a microscopic view of tin particles used in an inkjet additive process.

FIG. 18 shows raw resistance vs flex profile for 6 individual Methode ink flex sensors on Methode PET. A linear trend line is included. Variance is consistent as the sensors are bent between −90 to +90

FIG. 19 shows variance of sensors on Methods ink/PET across one batch. A 3rd order polynomial trend line is included.

FIG. 20 shows a computer generated model of an example spine sensing tape.

FIG. 21 shows normalised voltage to flex angle profile of Methode ink flex sensor pairs. A 3rd order polynomial trend line added for each sensor's raw data.

FIG. 22 is a breakdown of various subsystems of an example processing unit

FIG. 23 is an example PCB design with highlights. Multiplexer on underside of PCB. (Original image at FIG. 1)

FIG. 24 shows LTC4080 Typical Application Schematic

FIG. 25 shows an opAmp configured as a resistance to voltage converter. Requires negative Vref for positive output.

FIG. 26 shows a schematic of a half bridge Wheatstone and an instrumentation amplifier

FIG. 27 depicts creation of an instrumentation amplifier out of discrete opAmps

FIG. 28 shows an AD8236 Breakout

FIG. 29 shows a MCP42XX Digipot Pinout

FIG. 30 shows a Voltage Splitter TLE2426 Schematic

FIG. 31 shows a TLE2426 Pinout and Vi/Vo curve (Vi:Vin)(Vo:Vout)

FIG. 32 shows a ADXL345 Breakout

FIG. 33 is a high level representation of the firmware code structure according to one example embodiment.

FIG. 34 is a flowchart of a scheduler and a delay based system, of which the scheduler is used in SpineSensorV2A FirmwareVO 8 1.ino

FIG. 35 is a functional flowchart of readSensor( )

FIG. 36 depicts a visual explanation on how body overall curvature is obtained, and the body's orientation is ignored.

FIG. 37 Shows how the component vector a.b relates to c in terms of getting the angle θ in a consistent manner

FIG. 38 is a functional flowchart of calibrateSensor( )

FIG. 39 depicts overall Mapping of Atmega32U4 SRAM

FIG. 40 Shows how the stack and heap grows within an AVR micro controller

FIG. 41 Shows the front side and the backside of the bottom mount microusb SMD port in our final PCB.

FIG. 42 shows an example connection arrangement for serial USART in asynchronous mode

FIG. 43 shows a pinout according to the RN42 data sheet, as well a flatbed scanned underside dimensions of the pads

FIG. 44 Shows how SPI consist of a ring of shift registers with clock and chip select

FIG. 45 depicts addressing individual SPI devices with multiple CS# lines

FIG. 46 depicts cascading multiple SPI devices with a ring topology. All sharing same CS# lines

FIG. 47 depicts a typical SPI communication of a command and a byte being transferred before executing the command and value on rising edge of CS#

FIG. 48 depicts a Command Byte breakdown diagram

FIG. 49 depicts how to interpret SPI modes visually. First mode highlighted.

FIG. 50 shows typical wiring of an I2C device. Take note that there is a mandatory need for pullup resistors for SCL and SDL.

FIG. 51 depicts an ADXL345 register map

FIG. 52 depicts an ADXL345 POWER CTL register

FIG. 53 depicts an ADXL345 DATA FORMAT register

FIG. 54 shows S0 to S3 used as multiplexer channel selection.

FIG. 55 shows wireframe draft art of the User Interface for the PhoneGap Application, Left: Front-back flex. Right: Side to side flex

FIG. 56 shows an example PhoneGap architecture.

FIG. 57 shows a benchmark of popular jQuery syntax compatible libraries for .html and .class functions.

FIG. 58 depicts a canvas element. Measurement unit is in pixels. The origin, (0,0) is on the top left. The canvas is 500×375 pixels. The bottom right position is (500, 375)

FIG. 59 depicts a comparison of javascript canvas libraries

FIG. 60 shows Javascript and PaperScript scoping

FIG. 61 shows JavaScript, PaperScope and window global variables (data store)

FIG. 62 depicts an example calculation of the deviation from user-saved best posture position

FIG. 63 depicts an example 3D representation of the mobile application. Separate canvas for each curve.

FIG. 64 depicts an example Phone application process

FIG. 65 depicts a jsperf.com benchmark with many loop implementations is available. Looping performance benchmark.

FIG. 66 depicts example rendering: Left: No subpixel rendering (aliased), Right: Antialiasing is used to smooth the sprite as the origin point is not a set integer.)

FIG. 67 depicts First is the automated rig testing a flex strip. Second is the automated rig testing an optical break flex sensor.

FIG. 68 shows which ex sensor represents which subplot

FIG. 69 depicts an example multiplexer

FIG. 70 depicts an example microcontroller

FIG. 71 depicts example Power Systems

FIG. 72 depicts a RN42 Radio Module

FIG. 73 depicts a Zif Socket

FIG. 74 depicts an example Amplifier and Tuning System

FIG. 75 depicts an example Vibrational Motor Driver

FIG. 76 depicts an example Vibrational Motor Driver

FIG. 77 depicts an example Multiplexer

FIG. 78 depicts an example strip according to the invention

FIG. 79 depicts an example strip according to the invention showing horizontal displacement

FIG. 80 depicts an example strip according to the invention showing diagonal displacement

FIG. 81 depicts an example strip according to the invention showing vertical displacement

FIGS. 82-84 show examples embodiments in which a strip of the invention is utilised with different body portions.

FIG. 85 depicts use of one implementation of the invention comprising direct connection between a strip and a transmitter.

### DETAILED DESCRIPTION OF EXEMPLARY EMBODIMENTS

It is convenient to describe the invention herein in relation to particularly preferred embodiments relating to sensing posture and in particular by reference to spine position. However, the invention is applicable to a wide range of environments and situations and it is to be appreciated that other constructions and arrangements are also considered as falling within the scope of the invention. Various modifications, alterations, variations and or additions to the construction and arrangements described herein are also considered as falling within the ambit and scope of the present invention. Examples of such modifications include sensing the location in space and movement of other joints, such as a shoulder, knee, elbow, wrist, and so on.

Example Implementation—EWo3

This example provides a human Spine Corrective Device, consisting of a flexible tape that can be adhered to a person's back and which is calibrated to the user and sends wireless signals to a smartphone for recording and review. The device is easily applied with or without medical assistance to help self-correct spinal and postural problems.

This particular embodiment of the invention is directed to providing a cheap, easy to use and accurate alternative to monitor posture. The signal is sent from the device and displayed on the screen of a handheld device such as a mobile phone, for example using an App.

Various sensors can be used or interchanged as detailed herein but in one embodiment the device uses a custom sensor design with linearity and performance at a low cost.

The Ewo3 example is a consumer and medical device, that helps reduce the occurrence of spinal injuries related to lifestyle and work habits. This includes extended movements and static posture that would result in series of muscle strain and injuries over time.

Benefits of a device according to the invention include:
1. Accuracy—ability to inform the user with enough information to correct their posture.
2. Cost—cheap enough, similar to single visit to a physician
3. Motivation—Accuracy doesn't mean anything if the user doesn't follow the instruction, so the device incentivises the user to correct their actions with stimuli.
4 Reliability—users can trust the results, accuracy and improve their posture
5. Interoperability—The device ties in with other solutions in the space, eg. physicians

The device is aimed towards the consumer market instead of the medical market. This means increased emphasis on minimising cost. This particular example is focused more on preventing upper back injuries, over the lower back, as upper injuries being more common. This particular low cost example prioritises forward and backwards measurements over side to side posture, as most people know how to maintain side to side posture naturally.

Biofeedback is used for incentives and feedback (via long term graphs, medium term visual feedback by mobile phone app, to immediate feedback via haptic feedback alerts). Users can also message their physiotherapist or chiropractors for example in relation to their progress.

FIG. 2: Shows the breakdown of various subsystems of this example. It illustrates how overall system can be broken down into including;
Curvature of back
Flex Sensors
Accelerometer/Gyro
Data collection unit
Bluetooth communication
Smart phone with Bluetooth
Smart phone app with Calibration
Send data remotely to health professionals e.g. physiotherapist

Sensors

The overall system design revolves around a spine sensing tape. Each sensing tape's configuration consists of a number of sensors such that it can provide resolution to detect how much a person is bending (or slouching in the incorrect posture), both front-to-back and side-to-side. The size profile is slim so it is easy to apply without being invasive. The tape has been designed to be low cost and conform with existing manufacturing processes.

There are three types of sensor characteristics; unidirectional, bidirectional and bipolar. Unidirectional sensors will only change properties when bent in one of two opposing directions. Bidirectional sensors change properties when bent in both opposing directions, measuring only the magnitude of motion. Bipolar sensors change properties in both opposing directions, each direction yielding a different measurement.

A number of different sensing technologies may be used. Preferably the sensing tape comprises a resistive based flex array for forward back motion and (optional) accelerometers for enhanced functionality and accuracy.

Fiber Bragg Grating Sensors

Fiber Bragg (FBG) sensors consist of a specially made optical fiber which the sensing area is treated to selectively act as a mirror for certain wavelength of light. Straining the fiber creates a shift in the spacing of the grating, which can be detected as a change in light reflected from input light source, or its corresponding decrease in light intensity on the other end of the fiber). This is accomplished by periodically changing the refractive index of the sensing area of the core fiber. It has the advantage of very high accuracy.

Fiber Optics Sensors by Selective Abrasion or Breaks

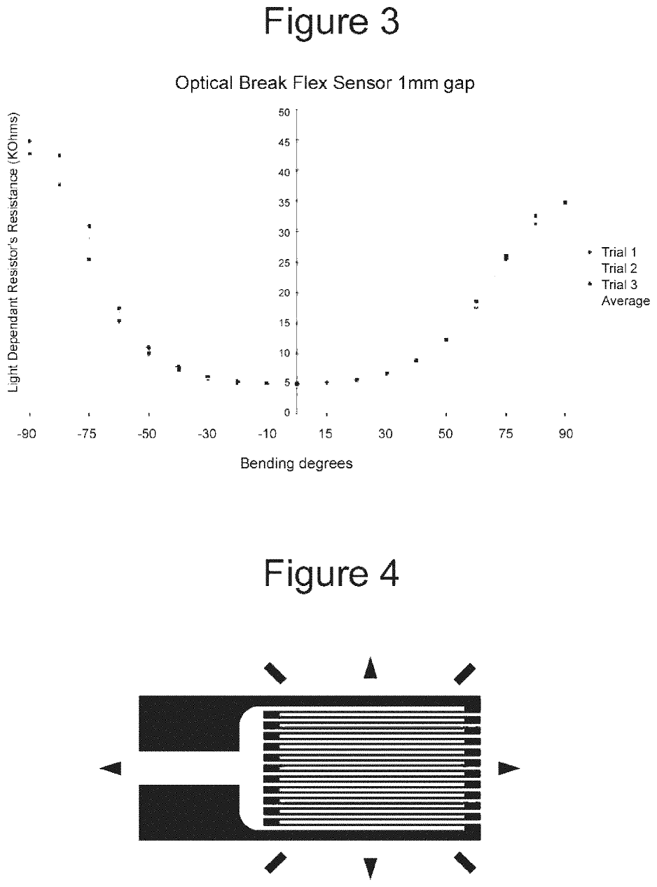

A break type of flex sensor may also be used in some applications. This involves a 0.5-1 mm gap cut in the optical fiber, covered over with heat shrink. A white LED on one end and a light dependant resistor on the other end, the automated rig reported these results in FIG. 3. This shows that the optical flex sensor and LDR combo is not linear, but has high repeatability over at least 3 consecutive trials. The data suggests this sensor is bidirectional, and cannot be used to detect forward or backward motion, only the magnitude of motion.

Micro Electromechanical Systems (MEMS)

MEMS are ICs that map mechanical changes, such as accelerometers or gyroscopes. MEMS are extremely accurate relative to their cost. They are often used in biomedical applications involving the human body. FIG. 3: shows the measurement of an optical fiber based sensor. −90 is left, +90 is to the right. An average of the trials is included in the chart.

Resistive Based Sensors

Bare Conductive paint is a non-toxic water based paint that is infused with carbon polymers. It has a resistance of 55 Ohms/Sq/micron, suitable for screen printing or painting.

Conventional strain gauges are passive devices that rely on resistance between fine filaments of conductive foil on an insulating flexible base such as polyimide. As the sensors are elongated, the cross sectional area changes as the filaments displace one another. The long parallel filament layout limits the direction to one dimension.

Strain gauge sensors are able to detect both flex and elongation, which make them suitable for this example embodiment. By integrating two columns of strain sensors embedded on the tape, it is possible to detect both forward and side motion.

Note: Ohms/sq(/micron) is the industry standard measurement for conductive inks. Sq., is defined as a ratio of length/width of the resistive section, actual measured resistance is subject to variance due to application methods (particularly/micron (z-direction, application thickness)

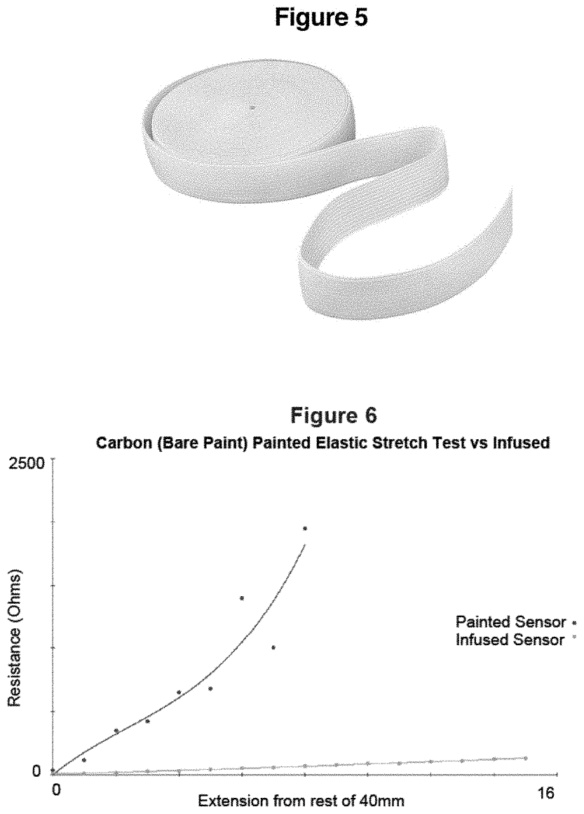

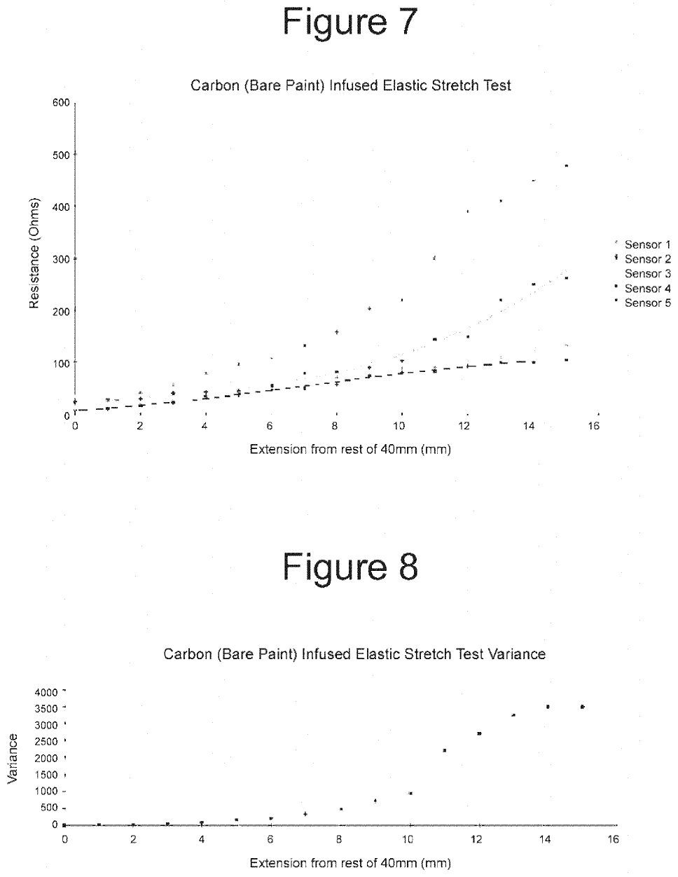

The characteristics of conventional strain sensors were replicated through the use of elastics fibres applied with Bare Conductive paint. (See FIG. 5: Interweaved elastic tape used for clothing). As the length of the elastic is increased, the distance between conductive carbon particles increases within the cross-braid structure. Elongation of the elastic fibres increases resistance when measured end to end. The braiding technique for the elastic conveniently limits stretching only in one dimension (lengthwise) which is analogous to foil based gauges. The resistance profile can be seen from FIG. 7, by integrating the strain sensors pre-stretched, bipolar sensing can be achieved.

Different application methods may be used; direct painting and infusing/soaking. Initially, a direct paint application onto the surface of the elastic produced a significant increase in resistance after a short change in length. By infusing the sensors with Bare Conductive paint diluted with water, more carbon particles are introduced and available for conductivity. From FIG. 8, there is significant variance between hatches.

Resistive Ink Based Sensors

These flex sensors are typically composed of a strip consisting of a resistive strip on an insulating material much like strain sensors, but do not require the same amount of manufacturing precision. The resistive ink is brittle, but is able to form a strong bond with the base material. As the sensor is bent, the insulating base within the localised area of bending will exhibit stretching which will pull the conductive particles apart and induce permanent micro cracks. This is the property that is exploited; further bends will open and close these gaps, resulting in a change in resistance with flexion. When at rest, the flex sensor returns to its intrinsic resistance (dependent on the shape of the resistive strip).

The flex sensor resistance varies depending on the radius at the flex location. The smaller the radius of a sharper bend the greater the resistance will be, due to micro cracks being further apart compared to a bend with a large radius, while it may also cause permanent damage. Furthermore, as the flex sensors are typically 2″ to 5″ in length, multiple bends along one sensor will give a unrepresentative resistance and flex angle. Thus, each sensor must limit bending exposure to one particular point while also maintaining a consistent bend radius for best accuracy.

Flexpoint, SpectraSymbol and Infusion Systems are manufacturers of commercial grade flex sensors and like most flex sensors on the market, they are all unidirectional sensors. Flexpoint claims there is no hysteresis associated with their sensors. It is worthy to note that the resistance to bend profile of the Flexpoint sensor is non-linear.

FIG. 6 shows Resistance vs elongation from 40 mm relationship of Bare Conductive painted elastic sensor. A sensor using the soaking method is added for comparison. Both data series have a 3rd order polynomial trend line.

FIG. 7 shows Resistance vs elongation from 40 mm relationship from one batch of Bare Conductive soaked elastics. A 3rd order polynomial trendline is inserted with each sensor.

FIG. 8 shows Variance of infused sensor stretch test. It is notable that variance increases significantly across the same production batch.

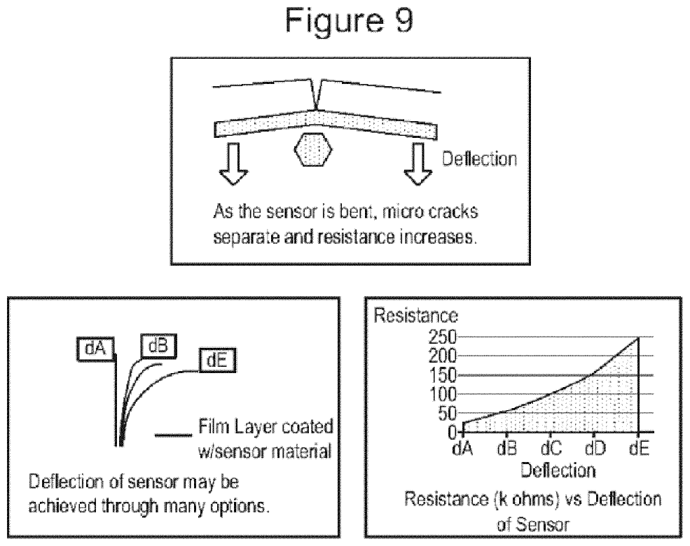

FIG. 9 shows that as the sensors bend, micro cracks appear ad the resistance increases. At localised area of bending, distance between induced micro fractures increase, increasing resistance of the sensor.

Resistive Ink Flex Sensor Development

SpectraSymbol FS Flex Sensor

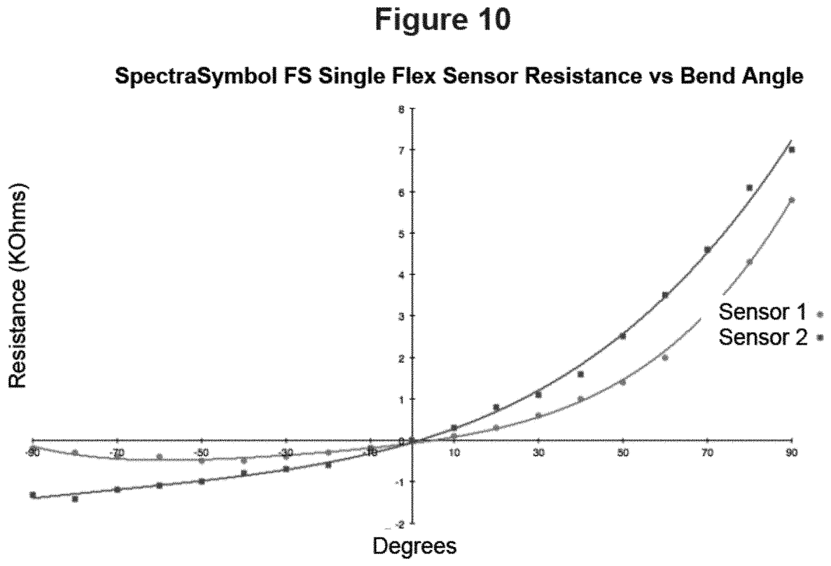

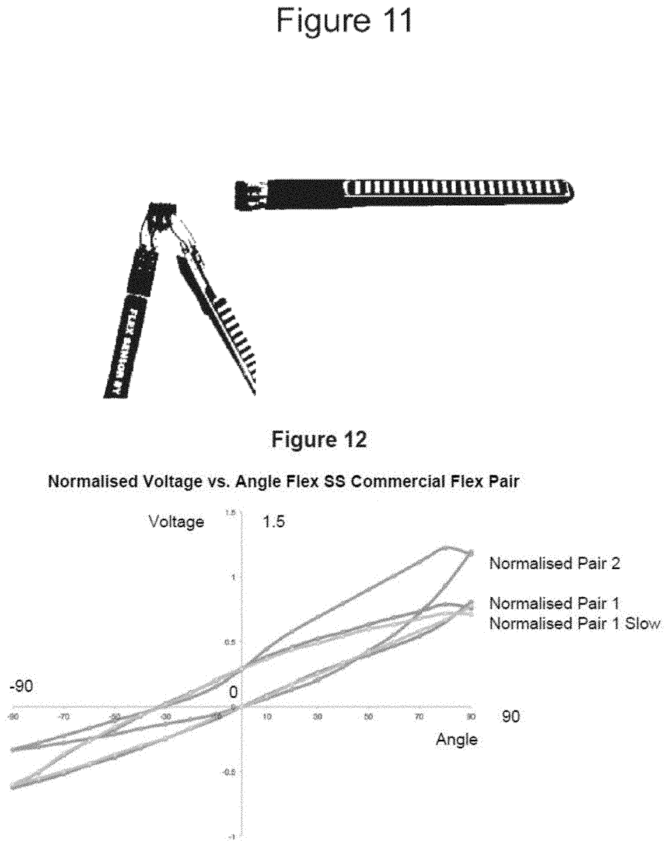

Commercial flex sensors by SpectraSymbol were used in one example. The sensor consists of a resistive element layered with conducting stripes, FIG. 11. The main element is the resistive component which changes due to flex. The conducting white stripes may serve the purpose of decreasing the intrinsic resistance of the sensor end to end. From FIG. 10, it can be noted that it is non-linear within the region of operation 0-+90 (away from the printed side). When bending into the region of operation, the length of the base material increases, thereby increasing the distance of each each micro crack. Flexing towards the printed side (−90) does not decrease resistance. This is due to the high density of resistive fragments; the distance cannot be decreased any further, thus resistance only changes by a minimal amount.

Since the SS flex sensors are unidirectional, one sensor alone is not enough to measure both forward and backward directions of motion. By placing two opposite back to back and securing with adhesive (FIG. 11, at least one of the two sensors will be in the active region of operation. The sensor pair is connected as a half Wheatstone bridge, used to measure the voltage difference with changes in angle flexion.

FIG. 10 shows Resistance vs flex angle profile of two individual SpectraSymbol 2.2″ FS flex sensors. Both data series are normalised by subtracting out the intrinsic resistance. A 4th order polynomial trend line is included.

FIG. 11 shows a SpectraSymbol 2″ FS flex sensor. The resistive element of the sensor is the black strip, with conducting elements placed on top to reduce resistance. Two sensors are arranged in opposite directions forming half of a Wheatstone bridge.

FIG. 12 shows Normalised voltage of SS flex sensor pairs. A sweep left (+)90 and right (−)90 results in hysteresis

Handmade Resistive Flex Sensors

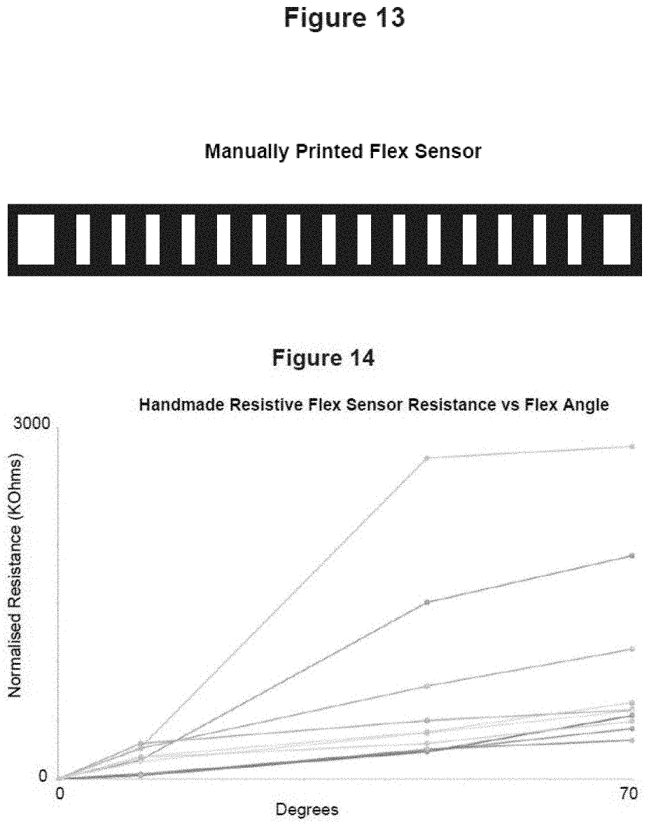

FIG. 13 shows an example handmade resistive sensor using BC paint, exible PET card and aluminium foil. In another example a resistive flex sensor was created by hand using BC paint, flexible PET card and aluminium for the conducting stripes. The resistance behaved similarly to the SS flex sensors. Due to manufacturing inconsistencies, there were some variations within the same batch of both intrinsic and operating behaviour due to uneven BC paint application (in the z-direction). While the range of resistance varies widely, 7/10 of the sensors created exhibited a similar change when subjected to flex.

Inkjet Printed Resistive Flex Sensors

There are many methods used for commercial manufacturing of flexible PCBs. Along with conventional semi-additive processing; screen and inkjet printing methods are given in this example. FIG. 14 shows Resistance vs flex angle profile of hand made resistive ink based flex sensors using Bare Conductive paint, aluminium foil on flexible PET card. Both data series are normalised by subtracting the intrinsic resistance.

Methods Developed for the Example Device

Semi-additive method is a common method to manufacture PCBs. DuPont's flexible polyimide based Pyralux is used as drop-in replacement for fiberglass backing. A solid ink printer is used to create the subtractive mask. This would only solve the issue of conductive tracks, not application of resistive ink.

Screen printing is an additive method used for large format poster printing. With the introduction of conductive and resistive inks, they are now commonly used in the industry for circuits involving customised shapes. Both Flexpoint and SpectraSymbol sensors are printed with this technique. The cost to set up each template mask is $200-300, however on-going manufacturing costs are minimal.

Inkjet printing is also an additive method using inkjet printing technology with special inks where the particle size is in the nanometre scale. Due to the required amount of processing, the cost of ink is very high, but does not involve additional setup procedures, therefore the number of different designs are not limited by cost compared to screen printing. As the device involves a lot of testing and prototyping, inkjet printing was the most suitable option for this example embodiment.

Most inkjet inks are water based, they contain other additives such as cellulose resin and humectant to stabilise the ink for storage and printing by controlling the viscosity to suitable levels (<20 cP) so it can be suspended within the print nozzle without leaking. Most importantly, colour pigments or conductive particles must be in the nanometre scale so as not to clog the print head. This means, diluted Bare Conductive paint (or from other manufacturers such as Conductive Compounds) cannot be used in an inkjet printer due to large particle size. Sonication is required through an ultrasonic bath to reduce size before it is fit for use.

Methode Electronics Conductive Ink

There are many providers of inkjet compatible inks in the market, such as InkTec, Methode Electronics and Plextronics. InkTec and Methode provide silver and carbon based inks, while Plextronics also produces organic polymer based inks used for photovoltaic and OLED circuitry. In this example embodiment, Methode ink is used

Methode PET are specially designed for Methode Electronics and their inks. Both have been treated to encourage ink drying and adhesion. Both PET versions are semi-transparent.

Printing Technique

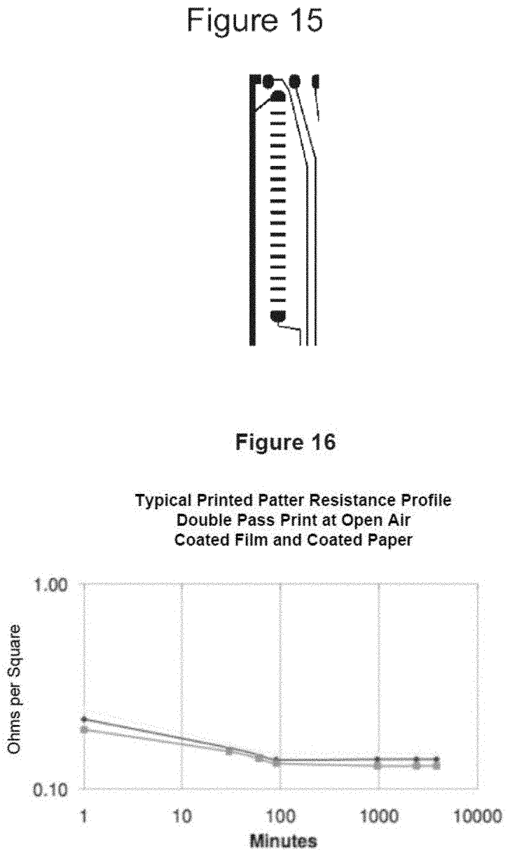

The sensors were printed using a 3-pass process. Firstly, a carbon layer is applied, followed by two more layers of silver+carbon on top. FIG. 15 shows Pass 1—Carbon, Pass 2 & 3—Carbon and Silver. A multiple pass technique is required to prevent any microscopic gaps between silver and carbon sections as the printer can only print one type of conductive/resistive ink at any one time, to maintain ink integrity. Using a layering technique, there will be at least be one continuous path end to end (through the bottom carbon layer). Conductivity decreases linearly each time a new layer (1 micrometre z) is applied, FIG. 16.

Alternative Flex Sensor Construction

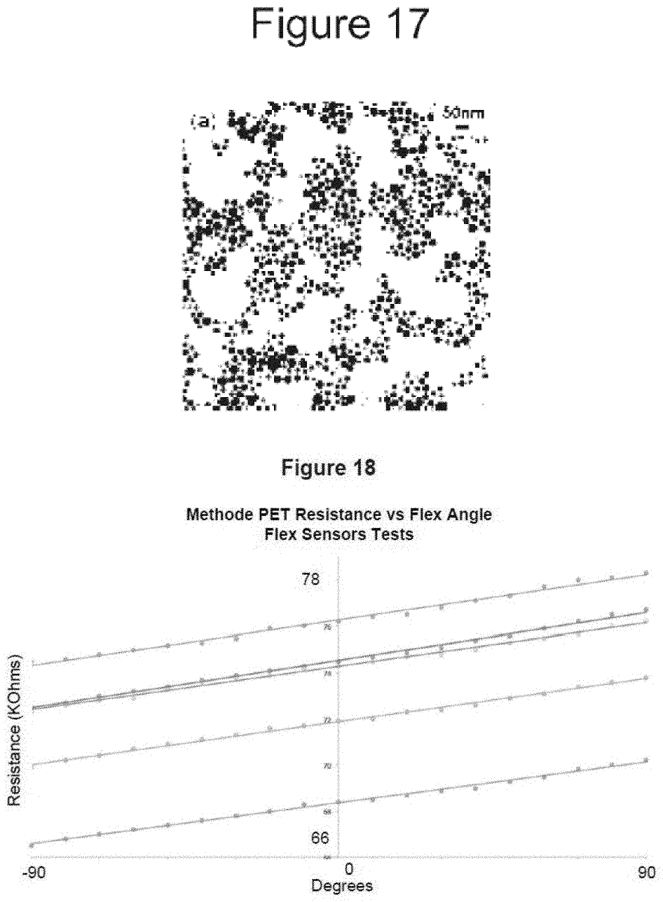

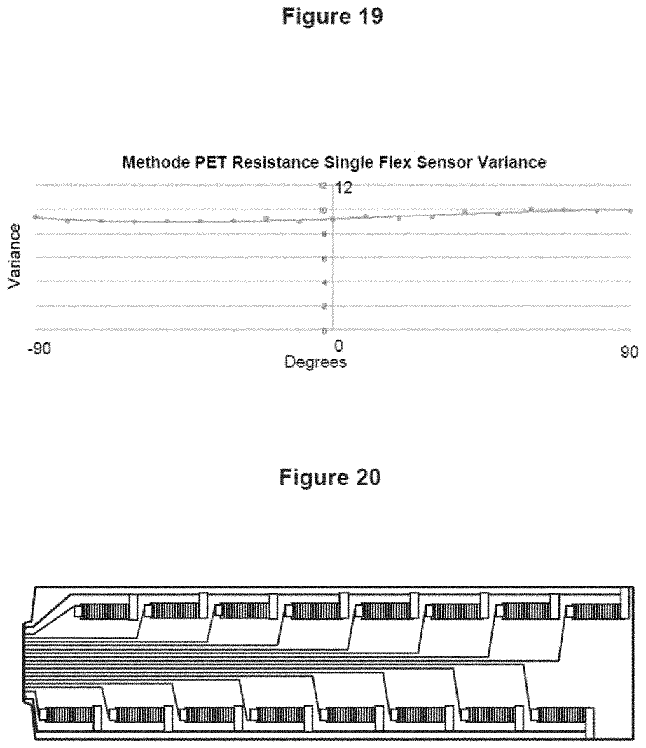

Sensors were designed and printed on both Methode PET and Epson polyester. Individual sensors were tested across a 180 degree range, from FIG. 18 there is very significant linearity across the −90 to +90 range. The data has not been normalised to showcase the minor variation achieved with inkjet printing. Variance is charted vs flex angle in FIG. 19; the amount is very minimal and consistent among the entire 180 degree range of operation. FIG. 16 shows single and double pass resistance curing in open air. FIG. 17 shows Microscopic view of tin particles used in a similar inkjet additive process.

Despite having a similar layout compared to the commercial flex sensors, we were able to create a bipolar linear sensor compared to the non-linear unidirectional characteristic. This is due to the low density of resistive particles applied by the print head. A microscopic scan of a similar inkjet application process from FIG. 17 shows gaps between each droplet. While these gaps are reduced and covered through multiple passes, this introduces spacing in the z-direction, creating microscopic distances between micro fragments. This gives additional room for particles to ‘travel’ as the material base is flexed in either direction. When bending towards the printed side, the material base reduces in length and reduces in distance, leading to a resistance decrease.

FIG. 18 shows raw resistance vs flex profile for 6 individual Methode ink flex sensors on Methode PET. A linear trend line is included. Variance is consistent as the sensors are bent between −90 to +90. FIG. 19 shows variance of sensors on Methode ink/PET across one hatch. A 3rd order polynomial trend line is included.

Despite already creating a sensor that is both bipolar and linear within the range of operation, we decided for this example to continue using the pair configuration analogous to the commercial flex sensor, (forming half of a Wheatstone) without significant changes to the existing circuit design on both the sensing tape and processing unit. The only cost is the amount of time associated with joining the segments by hand due to the constrained A4 size of the printer itself.

Each pair of sensors are laid out in a vertically cascading format to both increase resolution and reduce missing capture of bends (compared to end to end layout). The sensing tape consists of twelve pairs of sensors, each pair detecting for forward and backward directions of motion.

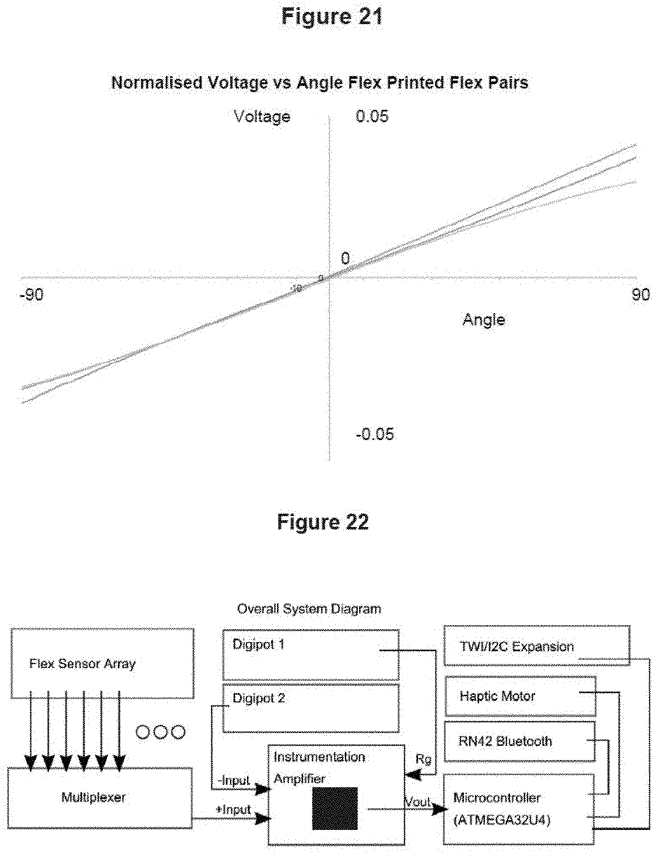

FIG. 20 shows a computer generated model of an example spine sensing tape. FIG. 21 shows Normalised voltage to flex angle profile of Methode ink flex sensor pairs. A 3rd order polynomial trend line added for each sensor's raw data.

Main VCC and Ground lines power the sensing tape sensors. The silver #9101 conductive ink has a significant resistance due to the size of the nanoparticles. To cover the long distance (y-direction), resistance is minimised by widening cross sectional area of VCC and ground lines (x-direction) and printing multiple passes (z-direction). Furthermore, due to the flaw of VCC and ground being placed along the side, induction is reduced by cross-connects at various points using conductive Kemo L100 silver paint. Pairs of sensors are joined using CircuitWorks silver conductive expoxy similar to a PCB via. The sensors use the vias as anchor points. There is no additional adhesive holding the pairs together to reduce any hysteresis caused by inflexibility observed with the commercial sensor.

Due to space constraints, the silver signal tracks have resistance in the range of 200 Ohms, however this is insignificant as the intrinsic resistance of the flex sensors are in the range of KOhms and the Wheatstone configuration. There is minimal noise due to the passive sensor design.

Additional expansion capabilities are possible with exposed I2C and power contact pads that transverse to the top of the tape where accelerometer clips can be attached. A 20 pin 1 mm width ZIF pad is printed directly using the printer that connects to ZIF on the processing unit.

PCB and Hardware Design

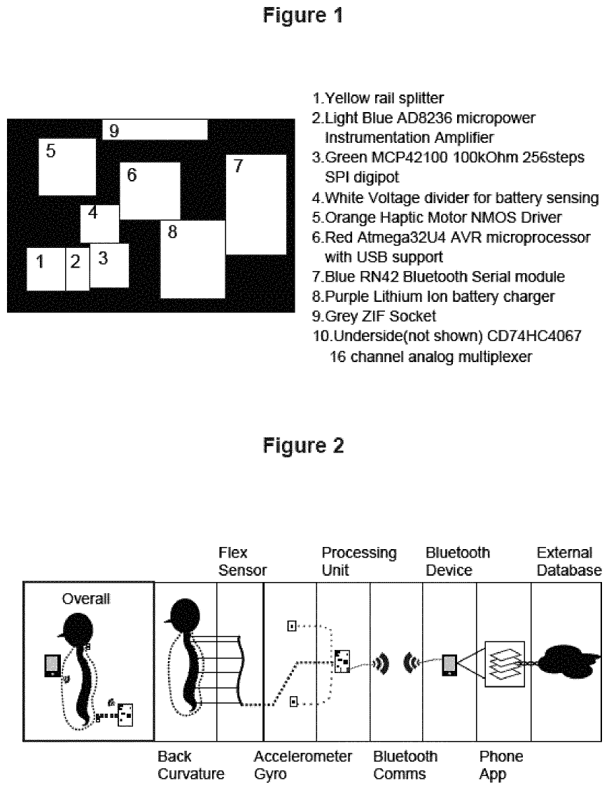

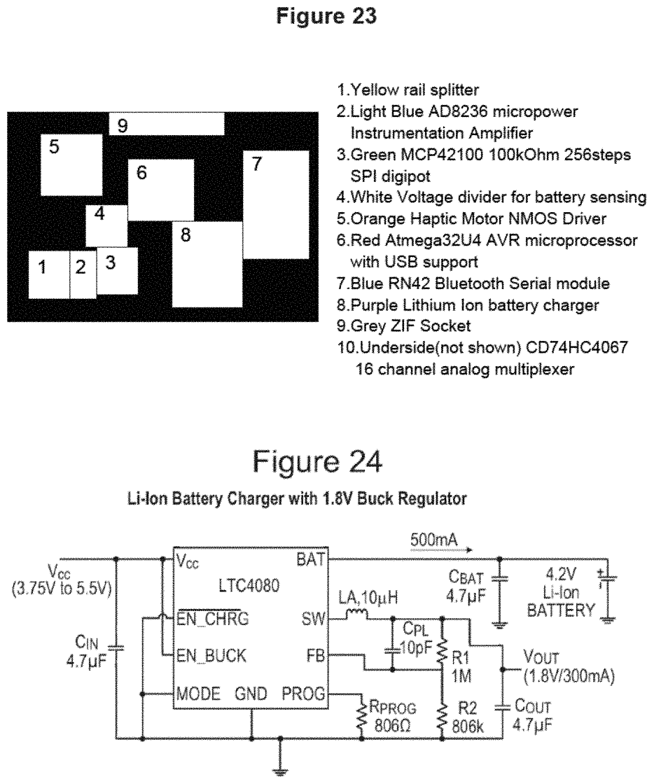

FIG. 22 shows the breakdown of various subsystems of an example processing unit. FIG. 23 shows Final PCB design with highlights. Multiplexer on underside of PCB. (Original image at FIG. 1)

1. Rail splitter (Outputs half supply voltage)

2. AD8236 micropower Instrumentation Amplifier

3. MCP42100 100 kOhm 256 steps SPI digipot

4. Voltage divider for battery sensing

5. Haptic Motor NMOS Driver

6. Atmega32U4 AVR microprocessor with USB support

7. RN42 Bluetooth Serial module

8. Lithium Ion battery charger

9. ZIF Socket

10. Underside (not shown) CD74HC4067 16 channel analog multiplexer

FIG. 24 shows the LTC4080 Typical Application Schematic

Power System

The device's power system was developed around the LTC4080 IC. It is a 500 mA stand alone lithium ion battery charger with 300 mA synchronised buck converter. It was decided to adopt the typical application schematic (FIG. 24) but modified to suit this example's requirements.

The ratio of the feedback resistance was changed. R2 in FIG. 24 was reduced to 315.78 kOhms. The buck circuit is triggered whenever the voltage drops below 0.8V at the feedback pin. Through a voltage divider place between the feedback pin and Vout, the ratio will allow for 3.3V at output to be seen as approx 0.8V to the feedback pin.

$$0.8 V = V out * \frac{315.78}{315.78 + 1000}$$

$$V out = 0.8 * \frac{315.78 + 1000}{315.78}$$

$$V out = 3.33 V rounded to two decimal places \text{\textbar}$$

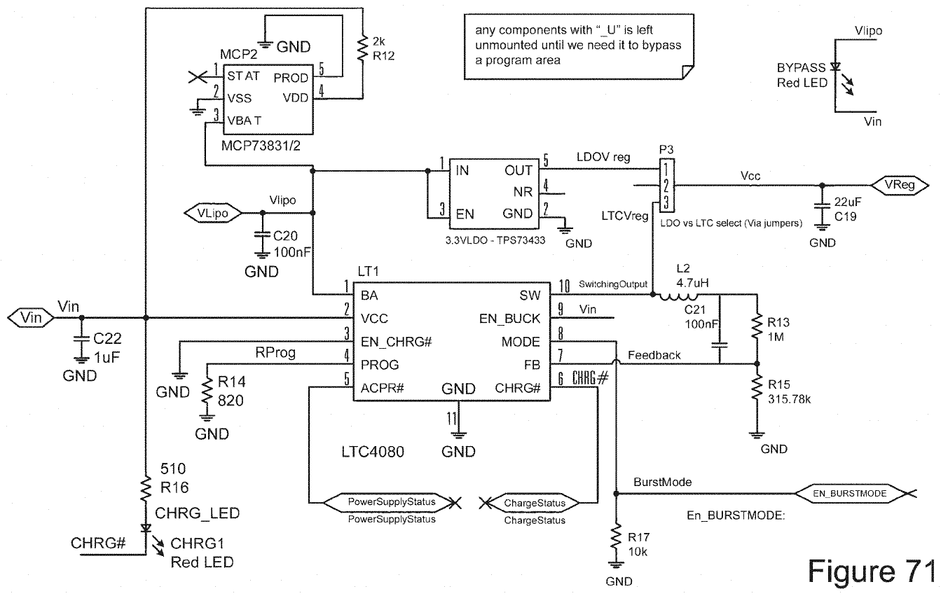

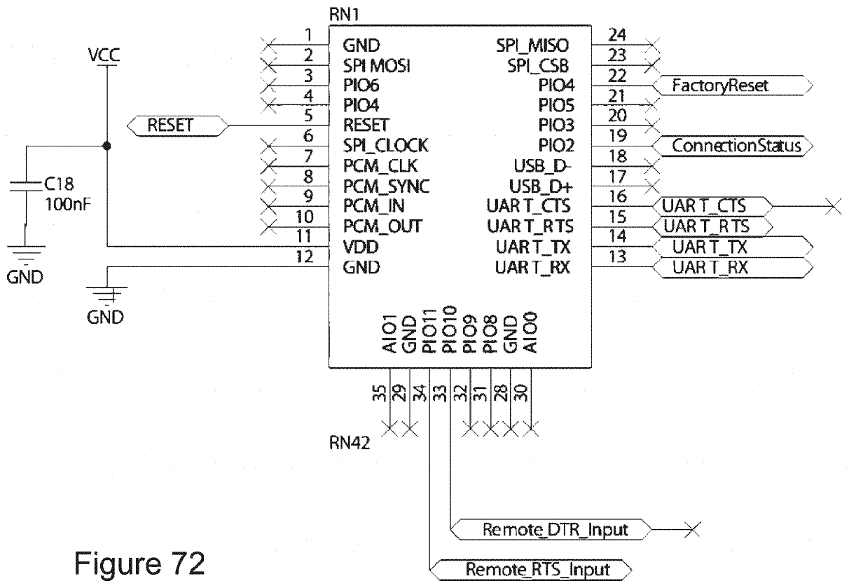

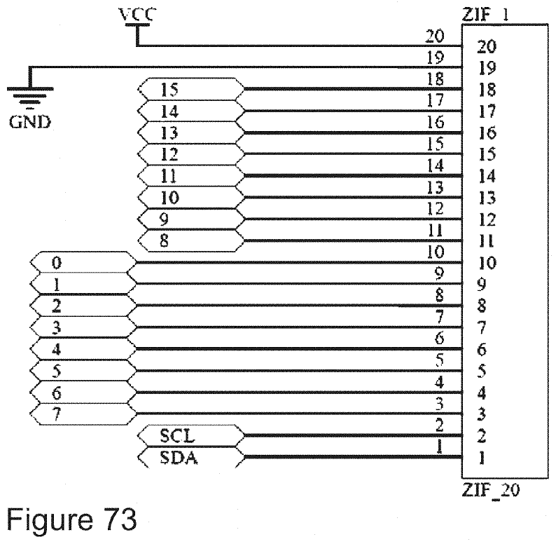

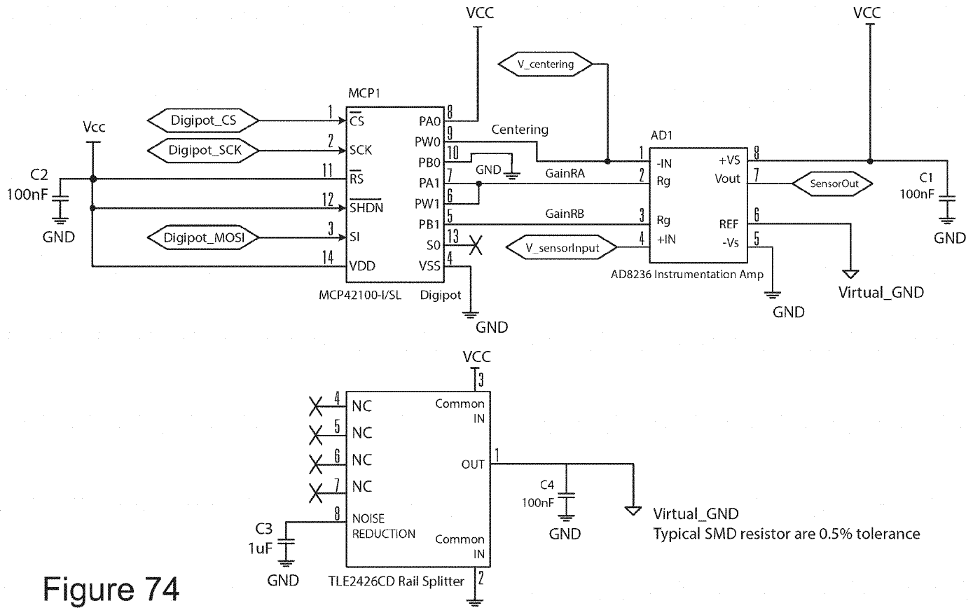

Refer to FIG. 71 for the schematic for the power system.

Pads for a fallback LDO and battery charger were placed on the PCB. Ultimately the LTC4080 did not work with the battery, until an extra diode was included. The diode was needed due to an oversight in the PCB design where EN BUCK was wired to USB power rather than the lithium battery. This meant that the buck was only functioning when connected to USB power, which defeats the purpose of incorporating it with a battery. Through the diode the EN BUCK at Vin can still be powered from the battery, but current from the USB is protected from charging the lithium ion cell directly (preventing a potential fire hazard). Measurement of the entire system power consumption from the final board. System Current is—

| | Sys Current | | Lipo (mA) | | Lipo (mA) | |
|---|---|---|---|---|---|---|
| Status | Draw (mA) | | (Via Buck) | | (Via LDO) | |
| 1st 5 sec | 30 | | 29.3 | | 46 | |
| Discovery | 49(Pulsed) | | 50(Pulsed) | | 56(Pulsed) | |
| Unconnected | 12.8 | 13.1 | 17.6 | 18.0 | 19.3 | 19.7 |
| Connected & Transmitting | 38.1 | | 38.6 | 40.9 | 41.5 | 42.5 |

The LTC4080 also has a burst mode, which would be useful if the system uses less than 10 mA, at a cost of increased rippling. (Buck Efficiency vs Load Current plot). While the corresponding PCB tracks for burst mode were routed, the system never drops below 10 mA, so this feature was not implemented.

The processing unit is currently designed for a 1000 mAh Li-Po from SparkFun. Assuming continuous Bluetooth transmission battery life can be calculated.

$$deviceRuntime ( h ) = batteryCapacity ( mA \text{/} h ) / consumption ( mA )$$

$$Using Buck converter \text{:} max \text{:} 25.9 hr = \frac{1000 mA \text{/} h}{38.6 mA} , \\ min \text{:} 24.4 hr = \frac{1000 mA \text{/} h}{40.9 mA}$$

$$Using LDO : max \text{:} 24.1 hr = \frac{1000 mA \text{/} h}{11.5 mA} , \\ min \text{:} 23.5 hr = \frac{1000 mA \text{/} h}{42.5 mA}$$

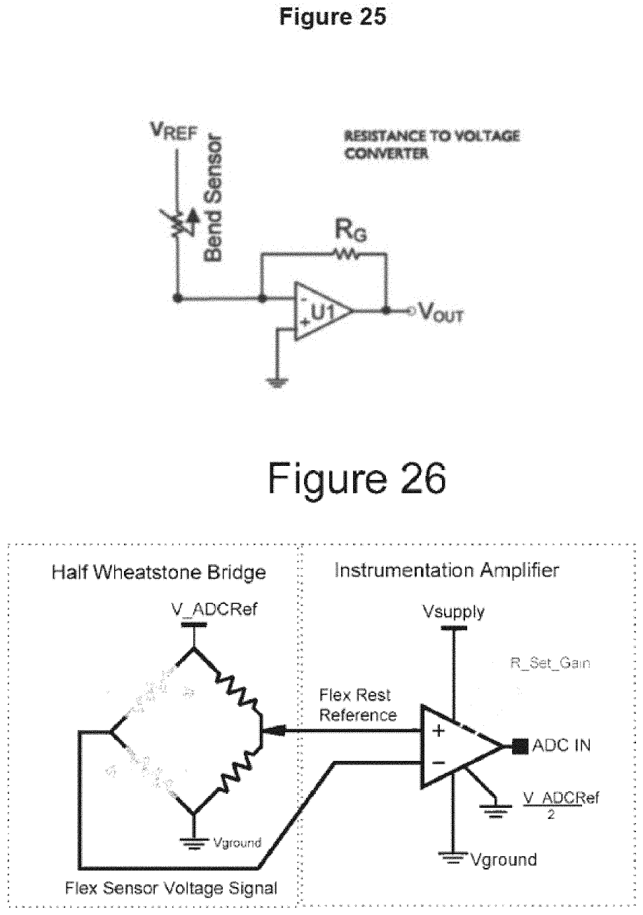

The runtime is an estimate. The difference in run time between the Buck and the LDO, is approximately an hour. The LDO is favoured due to cost and size. Areas to improve upon in current usage is the issue of data transmission rate. There is a correlation between Bluetooth transmission frequency and power consumption. As the user will not require real data, until when viewing the display directly, battery life can be improved by slowing down the flex sensor reading to once a minute. In some embodiments there is provided a sleep mode to minimise active wastage of power. FIG. 25 shows this opAmp is configured as a resistance to voltage converter. Requires negative Vref for positive output.

The battery or energy source for a unit according to the invention may be placed in any suitable position. In some preferred embodiments it is housed with the transmitter. In some preferred embodiments the battery is positioned within or adjacent to the same housing as the transmitter or a housing that is co-extensive with it.

Low Battery Sensing

Framework for low battery sensing was made by placing pads for two voltage division resistor to reduce the voltage of the lithium battery (voltage between 2.7 to under 4.5V) into a range that the ADC can sense with. Since accuracy is not important for this particular embodiment, two 1 MOhms resistors were used as sensing voltage divider ratio. This will reduce the voltage of the lithium battery to half, which is between 4.5/2=2.35V and 2.7/2=1.35V.

Signal Processing and Amplification

Since the observable changes in the printed flex sensor are very small signals, they require amplification before data can be acquired. This includes designs such as the resistance to voltage amplifier, shown in FIG. 25. In addition, the ADC signal input in the Atmega32U4 in the final design is configured as a single sided ADC input from ground to VCC.

Moving from a single sided to differential amplifier design, a Wheatstone (half bridge) circuit design was adopted. A Wheatstone bridge measures the difference in voltage via an instrumentation amplifier. In a half bridge design, one half of the bridge is a pair of flex sensor, and the other half of the bridge is a voltage divider providing the rest voltage reference. The rest voltage reference is the voltage seen by the flex sensors when straight.

FIG. 26 shows the schematic of a half bridge Wheatstone and an instrumentation amplifier

A test circuit was built to assess this concept. A breadboard was wired to the design in FIG. 26, where the rest reference voltage is represented by a hand adjustable potentiometer.

Instrumentation Amplifier Choice

Initially INA126 was chosen as the first instrumentation amplifier, from the datasheet there is a 0.9V voltage drop from the upper and lower rails. This means the range of swing the ADC will see in a 3.3V system is only 1.50002V=3.3V−0.9V*2.

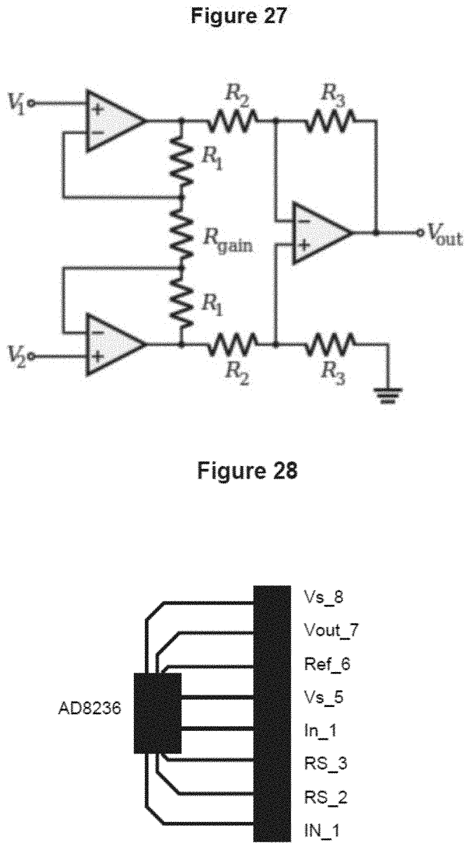

It is desirable to utilise the full range of the ADC and not lose 1.8V in sensing range. There was an investigation into the viability of building an instrumentation amplifier from LM324N. The LM324N consists of 4 opAmps with an output swing of 0V to 1.5V of upper supply rail voltage. It was wired as an instrumentation amplifier as shown in FIG. 27. As a workaround on the issue of upper rail voltage drop, the voltage supplied to the LM324N was at least 2V higher than voltage supplied to rail splitter (detailed in section 3.2.3) and the half Wheatstone bridge. 5V was supplied to LM324N and 3.3V was supplied to the sensors as well as ADC AREF(ADC voltage reference).

While the LM324N can be configured as an instrumentaton amplifier and a higher supply voltage, it was ultimately dropped in favour of Analog Devices's AD8236 shown in FIG. 27. This is because using the LM324N would require supplying two separate voltages, one for the LM324, and another voltage to power the flex sensors. The AD8236 has rail to rail voltage swing. Secondly the SOIC chip is smaller than the LM324N. Since the AD8236 is an integrated instrumentation amplifier, as opposed to the LM324N configuration, less discrete components are needed. Resulting in a smaller PCB.

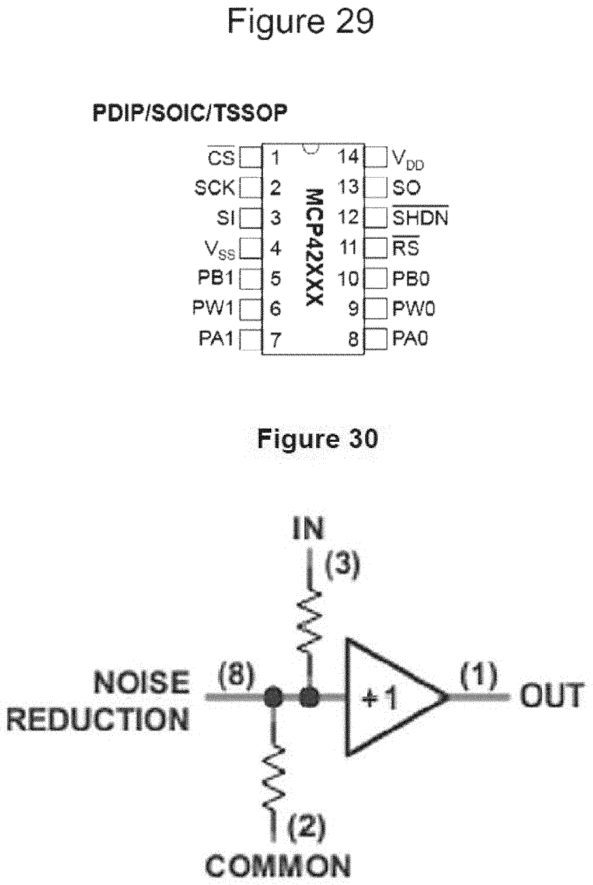

All the amplifier designs trialled used a single resistor to set the gain of the device. The AD8236 is similar in nature, the breakout in FIG. 28 shows the various resistance and the approximate gain that can be derived. To find the exact values needed to get a particular gain value, where RG is gain resistance, and G is the desired Gain value. Note there is a maximum gain of 200. FIG. 29 shows the MCP42XX Digipot Pinout.

$${R}_{G} = \frac{420}{G - 5} k Ohm$$

Digital Potentiometer

The primary role of the digital potentiometer in this design is to set the Voltage Reference as well as the Gain of the AD8236 Instrumentation Amplifier. The MCP42100 was chosen for this job, being a 100 kOhms digital potentiometer with two internal separately controllable potentiometers. The MCP42100 stores a value between 0-255 for each potentiometer. This means the theoretical resolution for this potentiometer is:

$$0.39063 k Ohms \text{/} Step = \frac{100}{256} k Ohms$$

The reason a 100 kOhm digital potentiometer was chosen is because the range of gain desired was between the gain of 10 to 200. This is since most flex sensors we tested with a typical swing around 0.1 to 0.3V. This requires gain of 33 and 11 respectively, to which a 100 kOhm digital potentiometer setting the gain, will be able to easily swing from gain of 10 to 200. This gives us plenty of leeway to eventually move to future sensors such as real strain gauges with minimal modification to the circuit.

For example in National Instruments white paper “Measuring Strain with Strain Gages”, there was a mention that strain gauges typically outputs less than 10 mV/V (per excitation voltage). This means for 3.3V, it can be expected to see around 3.3V*0.01=0.033V. To amplify such signals from these strain gauges (For a 3.3V system), the gain should be set to:

100=3.3/0.033

This should correspond to a gain resistance of 4.42K. And given the digipot has a step resolution of 0.39 kOhm, the gain resistance of 4.42K can be reached by setting the digipot resistance register to 11=4.42/0.39.

The other purpose of the digital potentiometer is to act as a voltage reference of the flex sensor at rest for the instrumentation amplifier to compare against.

As a voltage divider the digipot can split 3.3V into 256 steps for a step resolution of 0.01288V per step. Since in the digipot datasheet the typical “Full Scale Error” and “Zero Scale Error” for ‘V+=3V’ is typically at 0.35 of LSB which stands for LSB=V+/256 where V+ is the voltage supplied to the voltage divider. Thus LSB is the voltage step resolution calculated previously. Hence for 3.3V we can expect an error range of about:

$$-- 0.0045 = 0.35 * LSB = 0.35 * \frac{3.3 V}{256 steps} Voltage error$$

The digipot is set to 0.0128V*128=1.6384V , the real value is expected to be between 1.638437V and 1.638437V. Since the voltage changes in this example's printed flex sensors is typically no more than 0.1V, thls error is well below the error margin. This will be a different matter if dealing with strain sensors that require more accuracy.

FIG. 30 shows the Voltage Splitter TLE2426 Schematic

Rail Splitter and Virtual Ground

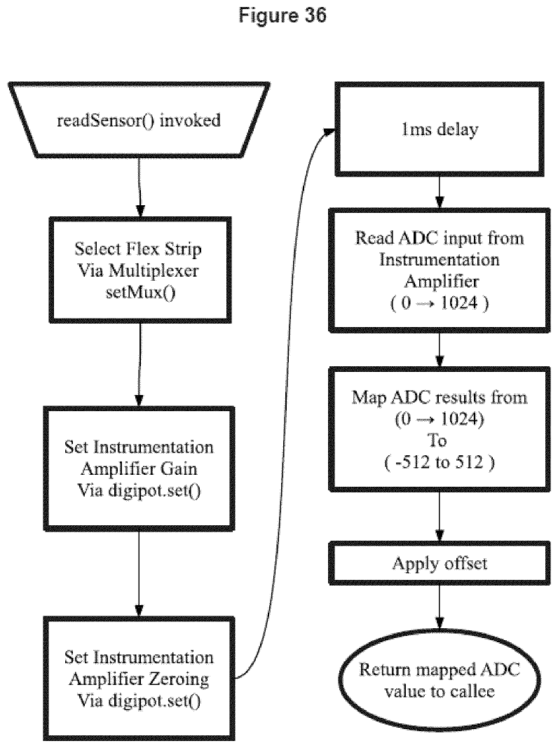

As seen in FIG. 36 the instrumentation amplifier requires a ground voltage reference. Since the Atmega32U4 ADC can only take between ground to the AVCC AREF, it means it cannot detect any negative voltage that would usually be seen if we set the instrumentation amplifier voltage to 0V. Setting the instrumentation amplifier to this new virtual ground. Allows sensing of negative voltage, as well as positive voltage.

The TLE2426 provides a high precision virtual ground operated at typically 170 uA with 5V input. This device output precisely half of the input voltage. The noise reduction pin was connected to ground via a 1 uF capacitor for further accuracy.

The rail splitter acts as a voltage divider where V out=V in/2. Since the accuracy of the voltage divider is based on the ratio of the two resistances, most rail splitters are limited by a tightly controlled resistance tolerance. An OpAmp config as buffer can be seen in FIG. 30.

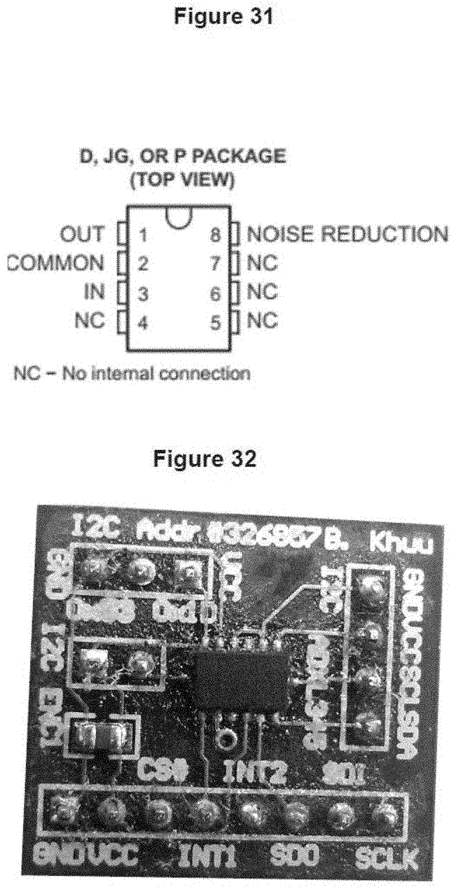

While the TLE2426 is designed to in-between 4V and 40V (As seen in FIG. 31), it works still when supplied with 3.3V. This is most likely due to the low current draw of the instrumentation amplifier's unbuffered ground reference input pin.

Multiplexer

The first design initially went for a 74HC4051 (8 channel multiplexer, 3 data/control lines), but later went for CD74HC4067 from Texas Instruments (16 chn mux, 4 data/−control lines) due to a need for more flex sensors.

External Communication

Bluetooth

In this example, Bluetooth was chosen over other wireless technologies such as ANT+ or WiFI due to widespread adoption for the standard Bluetooth protocol amongst the current smartphone user base compared to ANT+, as well as the relative low energy usage. We initially chose the HC-05, but switched to the RN42, both Bluetooth 2.0 module. Both modules can be interfaced to MCU by serial UART.

Expansion (I2C) and Additional Features

Accelerometer

In one implementation the MPU6050 is used. It has a motion processing unit to merge and fuse acquired data from both sensors, for greater accuracy. A custom breakout board for the ADXL345 was created (FIG. 32) which is a LGA (Land Grid Array) packaged accelerometer that had to be reflowed with a hot air gun. Once the ADXL345 was seated correctly, it hooks up to the I2C line. Its communication to the main processing unit is detailed below.

Haptic Feedback

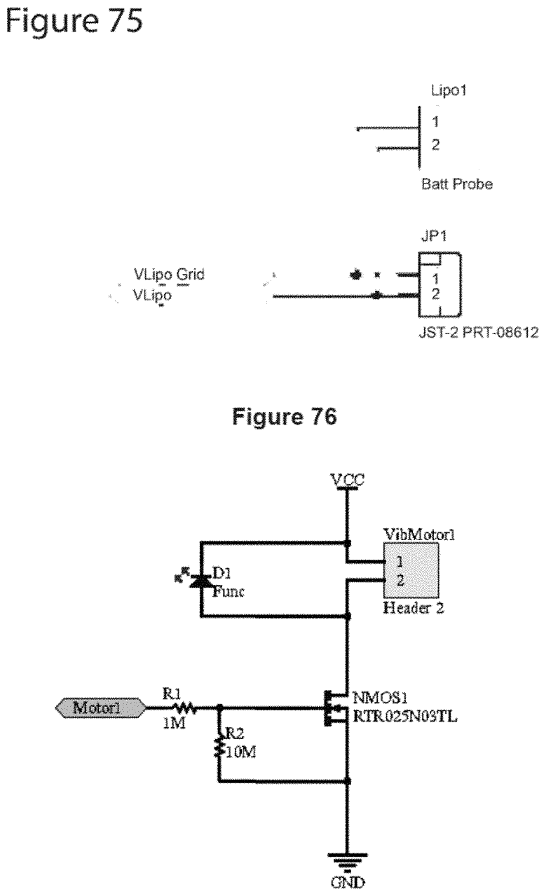

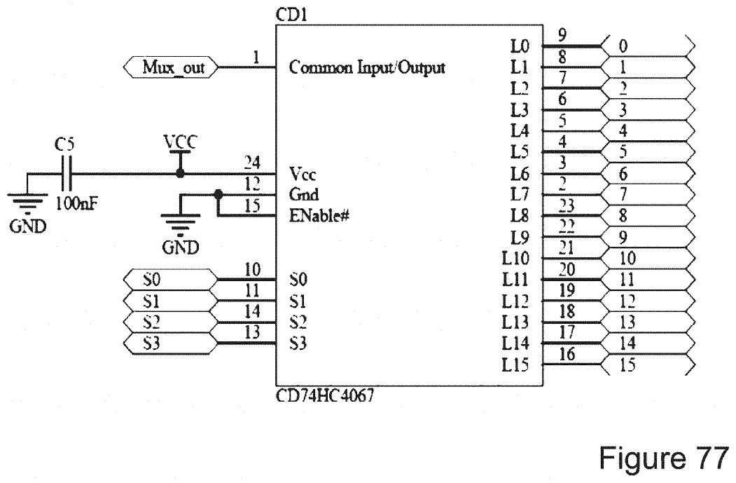

Haptic feedback was accomplished by an NMOS switch for a 3V haptic motor, a voltage divider is used to restrict the maximum current the motor receives, preventing burnouts. Also a diode is placed in reverse to power supply, to protect the circuitry from the vibrational motor's inductive kickback when shutting the NMOS gate. Refer to schematic at FIG. 75 for schematic of the driver.

Firmware Design

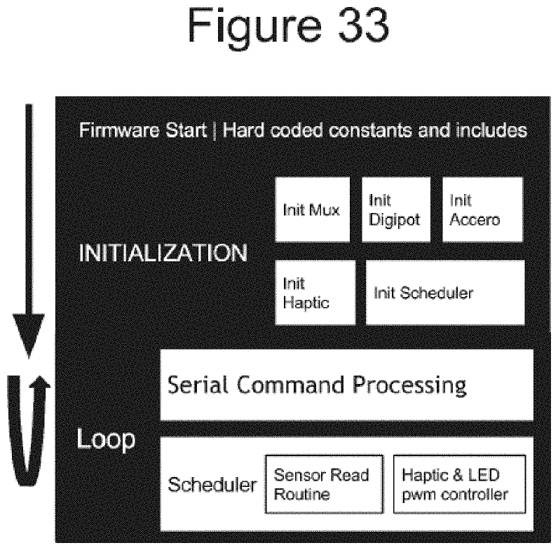

Firmwares is the code that is executed from the internal flash instruction memory of the Processing Unit's microprocessor. The microprocessor is based on the SparkFun Pro Micro 3.3V/8 MHz, an enhancement of the Arduino Pro Micro 16 MHz. The Atmega32U4 micro-controller contains an internal USB controller, used by the Arduino IDE for serial communication via USB. Overall structure of the firmware Arduino sketch is represented in FIG. 33 which shows all the major components and subsystem running inside the device.

Scheduler

Older versions of code consisted of an infinite while loop that performs a sensor read and then uses the Arduino delay( ) function to provide the interval between readings. The inclusion of the ability to wirelessly command the device to vibrate via the PWM sequencer required the use of a scheduler loop. This allowed the system multitask to a certain extent, by periodically checking and then running a particular function or routine after a certain pre-set amount of time has elapsed. Unlike the original delay( ) architecture, this has the advantage of preventing stalling other functions while a routine is waiting for the next round to run again.

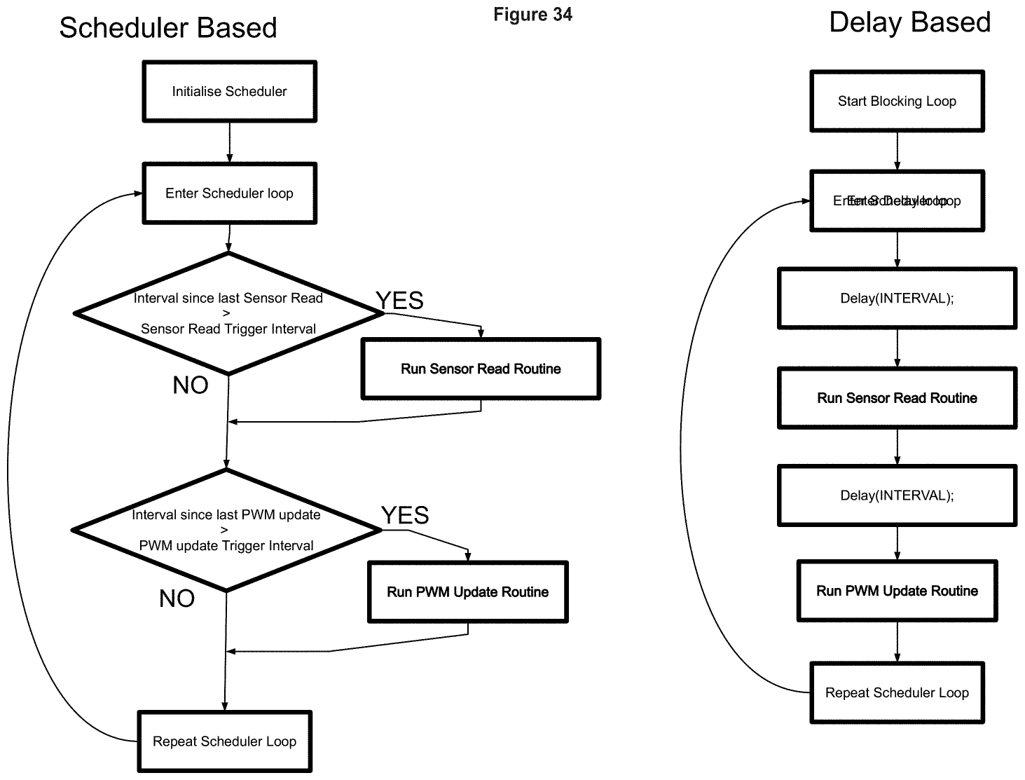

FIG. 34 shows how the scheduler code operates when compared to a delay( ) based flow. In the delay based flow, the time taken for the loop to execute is always INTERVAL*2. With the scheduler, the delay is minimal, only running routine functions when needed. The inclusion of the scheduler improves the responsiveness of the device.

Sensor Reading and Reporting

Incoming voltage signal from the flex sensor is defined to be at rest, when the signal is at half the ADC voltage reference from the Instrumentation Amplifier's output. As seen in FIG. 22, a sensor read routine will need to sequentially control both the multiplexer and the two digital potentiometer in order to effectively read all the flex sensors attached to the system. The example's firmware performs the reading and reporting of flex sensors in the device through a set of functions shown as follows;

int readSensor (int selectSensor)

This function outputs a selected sensor value after setting the digipots, multiplexer as well as applying the offset to the ADC sensor reading. Results from the function can be expected from −512 to 512, where the maximum ADC stepsize is 1024. If the ADC reads 512, then the output of readSensor should be 0, since the sensor is defined to be at rest when the instrumentation amplifier is at midpoint of ADC reference voltage.

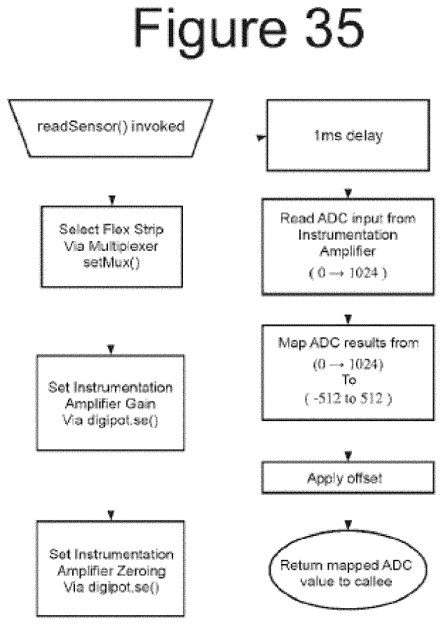

FIG. 35 sets out the steps taken in selecting and recording a flex sensor according to this embodiment.

The function, setMux( ) uses two arrays to control the mapping of parameter selectSensor to the multiplexer's pin channels. The mapping is cascaded as g_FlexConnectorMapping[g_FlexStripMapping[selectSensor]] and is used because the multiplexer pins on the PCB do not correspond to the ZIF socket on a one to one manner. In addition on the strip itself, the ZIF trace does not correspond to the sensors arrangement on the strip on a one to one basis. The reason for this, is due to the need to avoid overlapping traces in the PCB and the flexible strip. Also by keeping the mapping as two separate cascaded arrays, it allows for future modifications of flex sensor layout with minimal modifications to source code.

void readAllSensors(int *sensorsBuff)

This function uses a for( ) loop to sequentially iterate from the first flex sensor [readSensor(0)] to the last flex sensor [readSensor(TOTAL_MUX_CHANNELS-1)]. From the array pointer given in the function parameter sensorsBuff, the results from each readSensor( ) readings are inputted to its's corresponding position in the sen-sorsBuff[] array to return as the result.

void readSensorsDetails( )

This function checks the number of flex sensors and accelerometers enabled or de-tected, and prints it in JSON format for the phone app to use. It allows the phone app to know what to expect from the device and modify it's behaviour and display to suit the incoming data.

void readSensorsAsJSON( )

This retrieves the latest values from readAllSensors( ) and converts the integer range of readSensor( ) (−512 to 512) into a floating point representation of degrees. It also retrieves accelerometer values if available from the accelerometers on the I2C lines. Using the accelerometer raw x,y,z values, it converts these values into degrees for overall side to side as well as forward to backward bending. These values cane be sent via Bluetooth as a JSON string to the smartphone application.

Accelerometer Processing

Reading accelerometer values occurs in readSensorAsJSON( ).

The ADXL345 outputs three raw values x,y,z. These represents accelerations along the x,y,z axis. Since gravity is always constant towards the ground, it can be assumed that the readings on the accelerometers are component vectors pointing downwards.

The angle of interest for this example is the side to side flexing of the back which couldn't be done on flex sensors alone. This processing unit supports up to two accelerometers acting as tilt sensors. Two accelerometers are used, so that the actual overall curvature of the back can be obtained at any orientation of the body. This is by taking the angle from the accelerometer readings from the top, and subtracting from the Accelerometer reading from the bottom (FIG. 36).

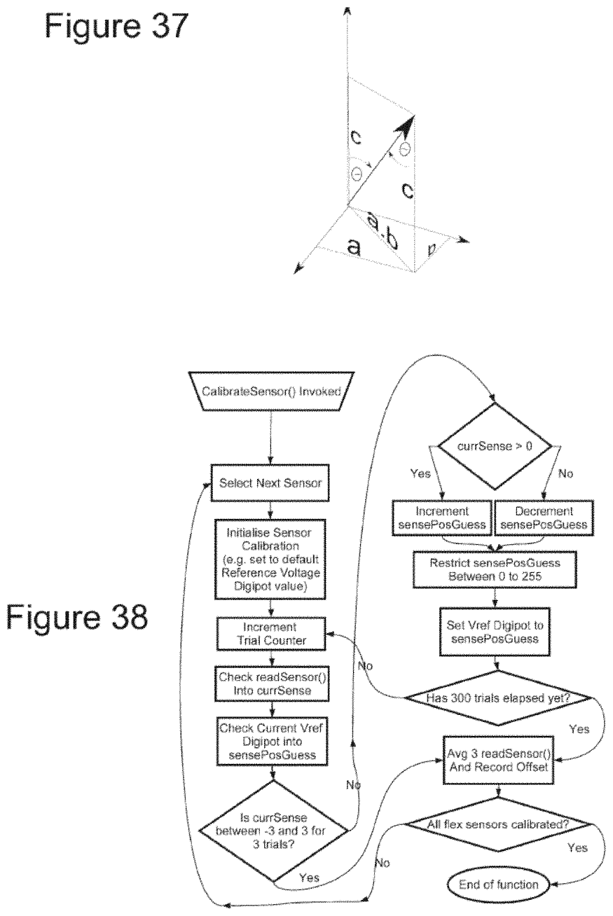

During testing, the ADXL345 breakout was working as expected for side to side motion, however data was inconsistent for forwards and backwards bending. This is due to an oversight, when calculating the side to side degrees, the original calculations took only two axes, when all 3 axis should be considered, e.g, x will not just decrease when bending side to side, but only forwards and backwards. FIG. 35 shows Functional flowchart of readSensor( ). FIG. 37 shows how the component vector a.b relates to c in terms of getting the angle θ in a consistent manner

// Original angle calculation. Faulty due to 2D thinking.

pitch=atan2(z, x);

roll=atan2(y, x);

Pythagoras's theorem (a2+b2=c2) should is used to find the component vector of X vs Y for Z or X vs Z for Y. FIG. 37 shows how this concept can be spatially represented.

// Amended calculations to take an extra dimension into account float xy,zy;

xy=sqrt(x*x+y*y); zy=sqrt(x*x+z*z);

Calibration

The calibration function in this firmware declared in the source as void calibrateSensor( ), mainly concerns itself with finding the right value for the reference voltage digipot; such that the voltage difference between the positive and negative input of the instrumentation amplifier, is 0V when the flex sensor is bent to the same angle it was calibrated at (Refer to flowchart at FIG. 38 for see how this function works.)

This function works by sweeping the reference voltage digipot towards centerpoint of the flex sensor using readSensor( ) as reference. Ref voltage incremented to match read sensor. This process is repeated, until both match up to 3 times or the maximum no. of attempt is reached (cannot calibrate). A convenient effect is that it is unlikely for this system to be able to lock on to a floating output. Thus we can use the effect of floating output when cutting the strip to detect the number of valid sensors. Alternatively a small pulldown on the sensor strip will allow for a default low voltage.

After a suitable reference digipot setting is found, the flex sensor setting is tested 3 times and the output is averaged. The average of 3 hardware calibrated flex sensor value for that particular flex sensor is used as a software offset for the strip in readSensor( ). The reason for this, is to make up for any hardware steady-state errors or offset that cannot be accounted for by the reference digipot, this is since the digipot resolution is only 0.01V (3.3V/255=0.01294). The flex sensor output variation from ADC reference voltage midpoint can often be smaller than 0.01V, and the amplifier can still amplify this difference.

On average it takes about 10 seconds to lock on to a sensing tape, but this varies depending on strip construction or delays between each trial. There are strategies to speed up the process. One of which, is to use a proportional control system, as opposed to an incremental approach to calibration. This speeds the process up by reducing the number of steps required to reach equilibrium, since a proportional control system will initially make large increments before gradually reducing it as it gets closer to the centerpoint. The simple incremental approach used from half of AVCC AREF as an initial guess. Most of the time the flex sensors will be around the midpoint of the ADC reference voltage, setting the digipot to midpoint reduces the number of iterations before calibration is achieved.

To improve accuracy, one might place two resistors on the upper and lower pins of the digital potentiometer, this cost the reference digipot its full range from 0V to VCC, but gains in accuracy in between. Alternatively consideration could be given to switching to a higher resolution digipot or a digital to analog converter (Since high resolution digital potentiometer can reach up to 10 bits, but a high end DAC can reach up to 24 bits.) FIG. 38 shows the Functional flowchart of calibrateSensor( ).

Ram Issues

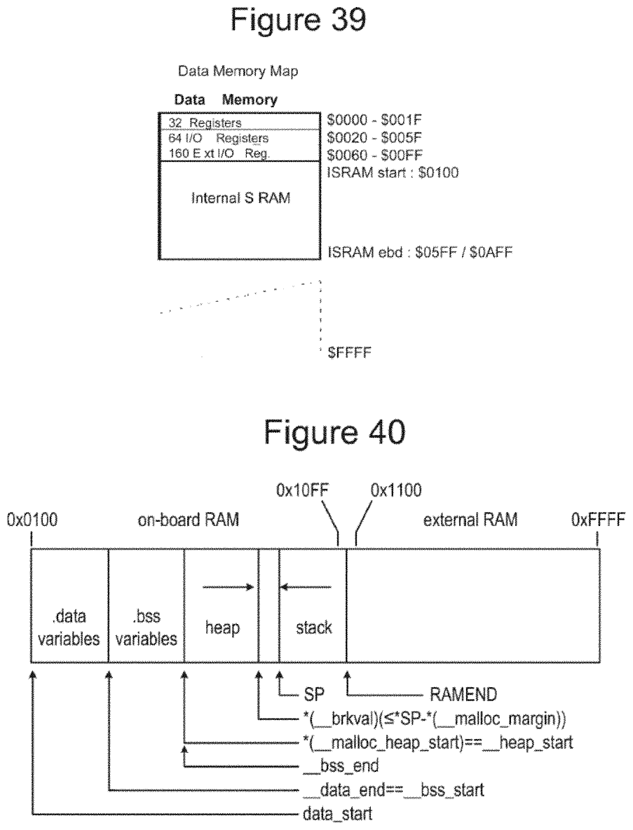

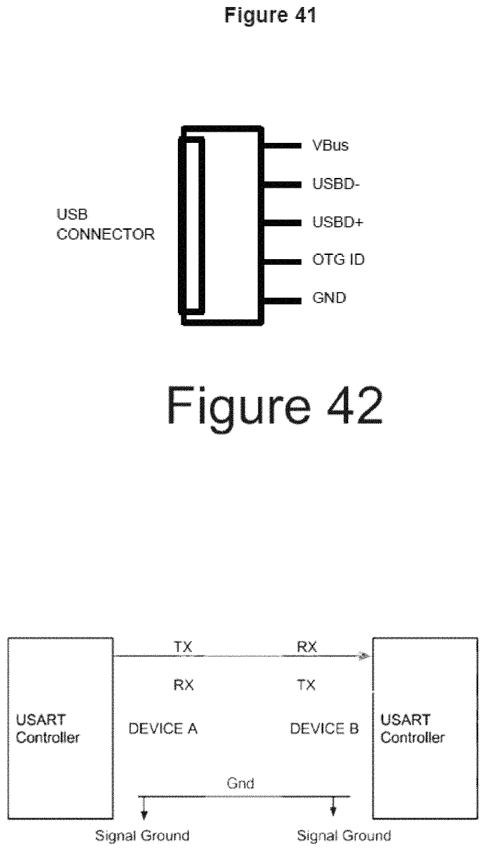

FIG. 39 shows the Overall Mapping of Atmega32U4 SRAM

The Atmega32U4 contains 2.5 KB of SRAM memory. Initially, code size was not an issue. However as code density increases, random crashes start to occur. There are similar processors on the market with a higher price range which solve the memory constraints.

Due to the choice of the Arduino platform, the debugging efforts were restricted to placing serial print statements everywhere. Suspecting overflowing RAM to be an issue, a ram check function was obtained from JeeLab and inserted in the initialisation section of the firmware as well as the standard loop. An issue with memory can be seen in FIG. 40.

Bare bone code showed that [memCheck]:2377, which is a big contrast to the 109 bytes of memory during the initialisation phase of the firmware. This indicates that the Atmega32U4 after the Arduino overhead, would provide only 2.377 kB of memory. This matches up with Jeelabs article, “The trick is to keep RAM usage low, because its a scarce resource: an ATmega has a mere 2048 bytes of RAM.” which is equivalent to 2.047 kB of memory. With this in mind, the firmware code was modified to reduce the number of strings that needs to be loaded to RAM before serial transmission.

Referring to FIG. 40, as variables are added and declared, the utilized memory grows in size in the SRAM towards 0×1100. At the same time, as more function calls are given, the stack pointer also grows in size towards 0×0100. In this example it was theorised that the wasteful use of print statements had likely caused the gap between the heap and the stack to grow too small that a stack—heap collision had occurred. This means that there is a possibility that either the heap or the stack had been partially overwritten, or corrupted. In the case of the heap, it means the scheduler will take an undefined amount of time to trigger the next interval. For the stack, returning to an address referenced from the top of a corrupted stack, would likely jump into a random instruction space instead of the original position the function was invoked from.

Annotated (via ‘»’) typical symptom of a firmware crash. freeRam( ) inserted to monitor amount of free memory space

>>> ‘...’ stands for omitted outputs
{ “fBA”:[94.22,98.61,110.92,68.38,−91.23,−78.93,−104.06,...
...
©SYSTEM STARTED@
“freeRam”:[109],
“freeRam”:[438],
{ “fTBA”:[−71.89,−71.72,−68.91,−111.45,−125.51,−100.55,−1...
“freeRam”:[427],
>> Program Crashed at this point
Code snippet from JeeLab, which measures amount of free RAM
int freeRam ( ) {
extern int _{——}heap_start, *_{——}brkval;
int v;
return (int) &v − (_{——}brkval == 0 ? (int) &_{——}heap_start :
(int) _{——}brkval);
}

Protocols and Communications

The hardware of this example uses a range of components which have their own requirements.

USB

USB is a highly popular connectivity standard for computers and devices that defines a physical connector, power, and protocol standards.

Physical Connector

An important aspect of the USB standard is to have a consistent physical interface or port so that there is a reliable way to connect the device to many other types of devices. In this example we use the ubiquitous micro USB port which is found in many consumer devices. Below is the standard USB pin configuration

| Pin | Name | Cable Color | Description |
|---|---|---|---|
| 1 | Vcc | Red | +5 VDC |
| 2 | D− | White | Data− |
| 3 | D+ | Green | Data+ |
| 4 | OTG ID | None | OTG Slave/Host Select |
| 5 | Gnd | Black | Signal/Power Ground |

USB Power

The main selling feature of USB is that it is one of the few standards that carry power along with signalling, unlike the older serial and parallel ports. According to the USB standard, only 5V can be supplied

A major reason for choosing micro USB versus the barrel plug or other standards is the ubiquity of USB charging. This essentially means that users will be unlikely to ever be out of a port to charge their devices. This reduction in proprietary sockets reduces the amount of E-waste.

USB also allows new code to be readily uploaded and serial console access over USB. These features allow for rapid prototyping by removing the need for an external programmer beyond loading the Arduino bootloader. In addition serial access over USB also allows for future upgrades for end users, to enable access to new accessories on the I2C bus or to fix bugs within the firmware.

UART and USART

USART stands for Universal Synchronous/Asynchronous Receiver/Transmitter. UART is Universal Asynchronous Receiver/Transmitter. A distinct difference between this communication standard and other wired standards such as I2C or SPI, is that USART and UART do not require a dedicated clock signal to signal the arrival of a new bit. Instead the clock is determined by the speed of the data transmission itself. This speed is determined by the matching baud rate that is set separately at each end of the connection, any mismatch in baud settings could lead to data corruption.

USART defines a receive pin as RX and transmit pin as TX. Thus for full duplex com-munication, you need to connect the TX of one device to the other device RX pin, and vice versa. Refer to FIG. 42 for an example of the connection arrangements.

The Atmega32U4, has a peripheral feature described as ‘Programmable Serial USART with Hardware Flow Control’. USART also has the capability of transmitting a clock signal as master or receiving a clock signal as slave, this synchronous mode allows for faster transmission compared to asynchronous mode. Since transmission speed is minor, the USART in this example is configured to operate in asynchronous mode only.

The example's device micro controller is connected by USART to an external Bluetooth module. The voltage output of the TX corresponds to the rated input of the other devices RX. Overlooking this requirement runs the risk of burning the RX input of the UART device on the other end. The baud rate settings, parity, and flow control mode should be the same on both sides.

EWo3 device communicates over USART in 9600 baud speed with no hardware flow control.

In Arduino, the baud rate for Serial object is set by this function Serial.begin(BAUD_RATE), where BAUD_RATE would be typically set to a value of 9600. It is important when you select a higher baud rate, that the oscillator on both ends are accurate enough. This means it is not possible to use the internal RC oscillator of the Atmega32U4 without calibration.

To send a string of characters over USART via Arduino, type:

» Serial.print(hello); // Prints a string over serial

» Serial.print(hello); // Prints with new line char

Bluetooth

Bluetooth is a wireless communication technologies for short range personal area networking with local low powered electronic devices. It is often recognised for its usage as mobile phone wireless headset.

In this example, Bluetooth is used as a convenient way to connect the processing unit to the smartphone. There are two versions of Bluetooth as of 2013, Bluetooth Standard, and Bluetooth Low Energy (BLE). BLE will be the primary choice in the coming years, despite its lower data transmission capability compared to standard Bluetooth, due to its lower power consumption and ease for the user to connect to such devices.

However even though BLE is the better choice for this application, this protocol has no default Serial Profile (SPP) that is found in standard Bluetooth, and our priority lay higher for a simple and reliable communication protocol as opposed to a device with longer run-time (due to better energy efficiency).

Roving Networks RN42

The RN42 is a Low power Bluetooth transceiver with 26 uA sleep, 3 mA connected, 30 mA on transmit mode. This is marked improvement from the HC-05 standby connected current of 8 mA at minimum, to the average current of 25 mA.

The RN42 uses a different baud rate of 115200 which is extremely fast. To reduce the baud rate, the easiest method to do this is to set the pin 2 (which corresponds to GPIO7) for the RN42 to force the board to function at 9600 baud. However this comes at a cost of future flexibility of allowing for software reconfiguration of the baud rate to a higher level such as 115200.

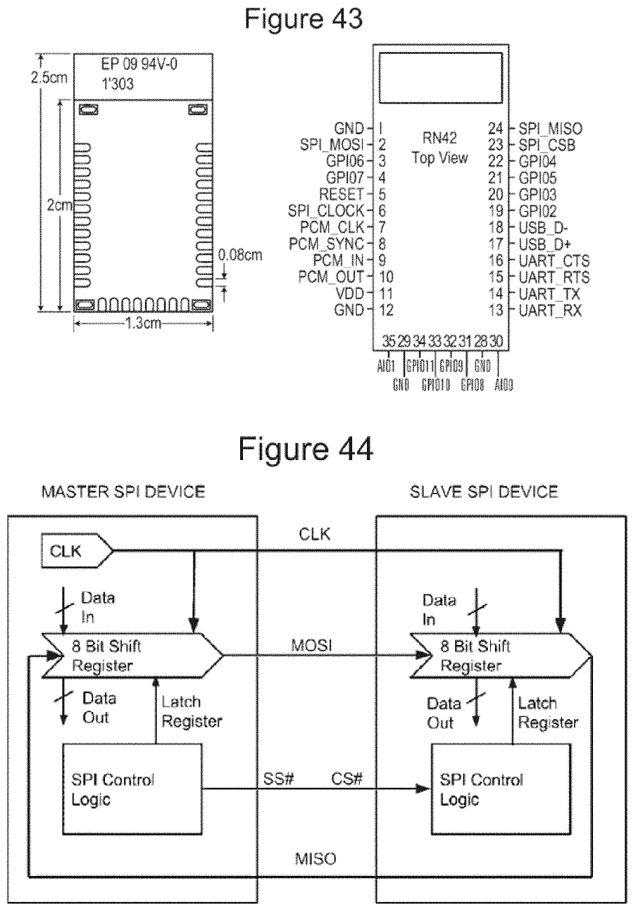

FIG. 43 shows the Pinout according to the RN42 data sheet, as well as flatbed scanned underside dimensions of the pads

A second method is to add code which upon reset of RN42 (which will provide a 60 second window for entering configuration mode), will send configuration commands to the RN42 in a non permanent way.

The last method is to manually enter configuration mode as for the second method, but instead of issuing a temporary command, a persistent SET command is available. This was what was used for the final PCB due to technical difficulties in getting temporary settings to work. FIG. 44 Shows how SPI consist of a ring of shift registers with clock and chip select.

Command set references;

SU,<rate>

Baud rate, 1200, 2400, 4600, 9600, 19.2, 28.8, 38.4, 57.6, 115K, 230K, 460K, 921K, only the first 2 characters are needed. EXAMPLE: SU,57 sets the baud rate to 57600 baud, SU,96 is what we used to set our breakout to 9600

S-, <name>

Serialised Friendly Name of the device, 15 characters maximum. This command will automatically append the last 2 bytes of the Bluetooth MAC address to the name. Useful for generating a custom name with unique numbering. Example: S-,MyDevice will set the name to MyDevice-ABCD these are the two command most likely sought after, if seeking to persistently set the RN42 for a particular application.

SPI (Serial Peripheral Interface)

A SPI Bus is a simple serial interface designed for communication between ICs, such as a microcontroller to a digital potentiometer.

FIG. 44 illustrates a generic view of most SPI devices. The actual internal circuit and how each device reacts to Chip Select CS# may differ. It is thus very important to consult the datasheet for exact specification of how to communicate to each chip.

The processing unit digipot supports an SPI interface. It is best visualised via the above diagram for reference, as two shift registers connected as a ring memory structure. To transfer a byte from master to slave, each bit is sequentially shifted from one set of shift register to another shift register on each CLK(Clock) cycle until all the bits are in the slaves shift register.

For this particular digipot (MCP42100 dual 100 kOhm SPI digipot), it uses the Chip Select pin to determine when to execute the next command. To which the digipot will set its resistance only after shifting all bits from the microcontroller to the digipots shift register and then having its CS# pin go high to let the digipot know that its not to drive the MISO line anymore (The MISO line will be tri-stated so that the bus is free for other SPI devices to use again).

CS#: chip select

Lets the slave chip knows if its being talked to. The slave MISO pin is disconnected upon CS pulled high.

CLK: Clock

Clock signal to indicate that the next bit is ready to be shifted in

MOSI: Master out Slave In

This pin allows a slave chip to receive bits shifted into it, on very CLK edge (Rise or fall depends on spec)

MISO: Master in Slave out

This pin allows for the slave to shift bits out towards master, on very CLK edge.

It is quite possible to leave out MISO, if the IC in question. But only if the master is never ever expecting to hear a reply from the slave. This is the choice made for this example, for SPI communication to the digipot which is merely an output device and thus we had no need to know of its current state.

Addressing multiple chips in SPI: below are the two most common methods of interfacing with SPI. There is also a third method called mSPI.

Chip Select

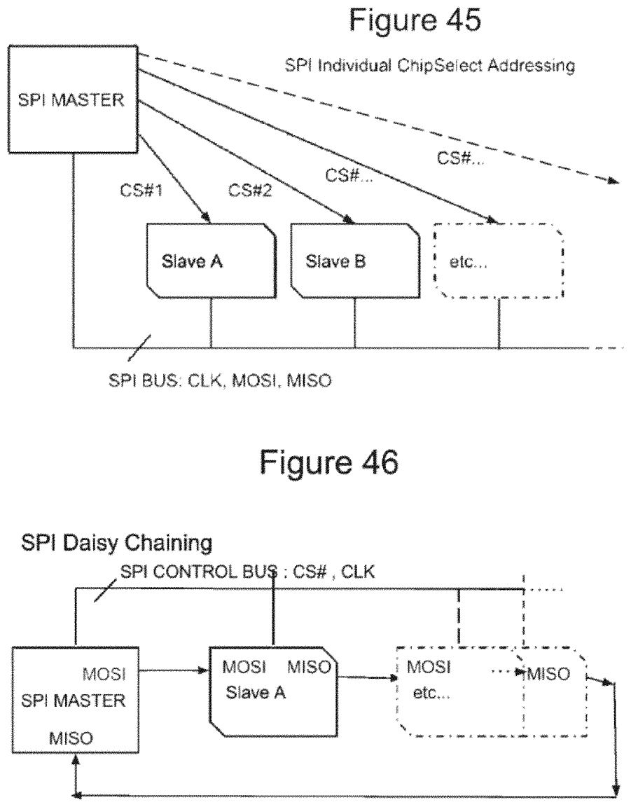

In this scheme, all the SPI slaves are connected to a common SPI BUS (CLK, MOSI, MISO), and each slave is addressed by a separate Chip Select line from master as shown in FIG. 45, More than 4 slaves would require a digital multiplexer (with negated output since CS# is active low) where the first pin is not connected so that all slaves can be disconnected.

Daisy Chaining

If time is not a critical a factor, and PCB routing space is at a premium, there is an option of daisy chaining the MISO of one chip to another MOSI input as seen in FIG. 46. This does mean that if each slave shift register is 8 bit long, then adding additional slaves to the SPI line would in theory add an additional linear amount of time.

MCP42100

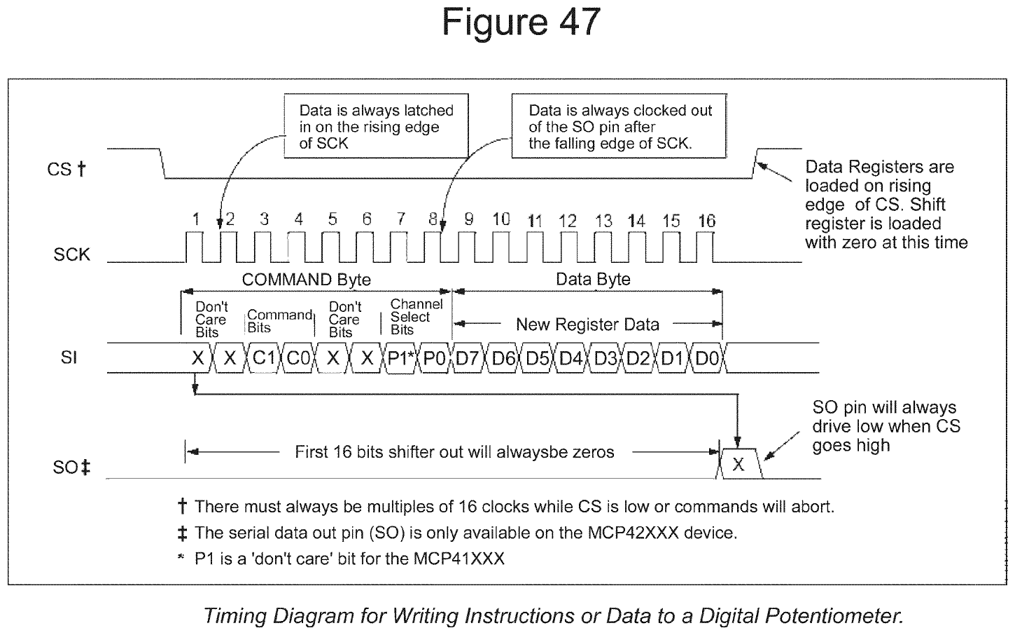

The MCP42100 dual 100 kOhm digipot is an SPI device that has its own command specification shown in its datasneet. Commands to the digipot take the form of two bytes (byte=8 bits). As shown in FIG. 47 first byte represents the command to be executed (Command Byte), and the second byte represents the value to be used in a command (Data Byte).

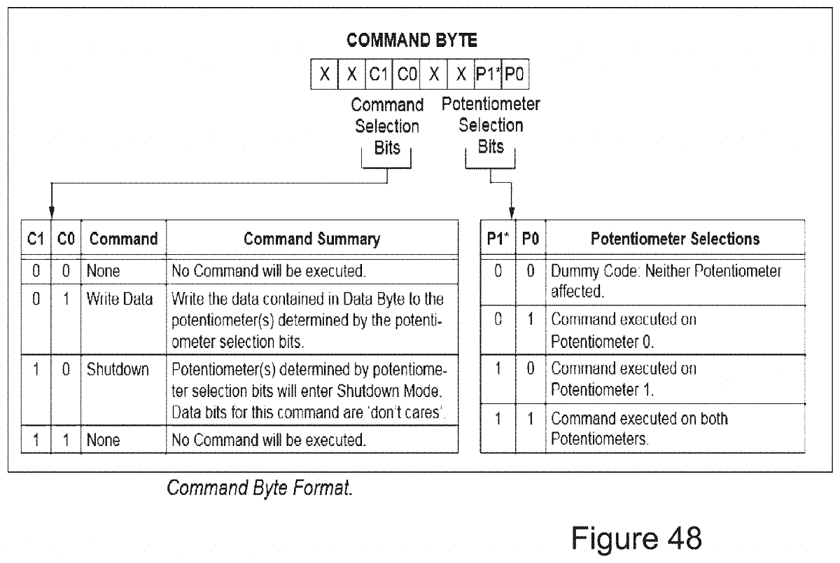

FIG. 48 points out that in addition to a write command, it is possible to have a shutdown command which will disconnect a digipot channels pinA, and short B and C together.

These are the Diaipot write commands in binary. Of which these combinations can be made:

| Description | Command Select | Pot Select |
|---|---|---|
| Write to pot 1 | 0001 | 0001 |
| Write to pot 2 | 0001 | 0010 |
| Write to pot 1 and 2 | 0001 | 0011 |

Communicating to the MCP42100 via bit banging

While researching on how to communicate to the MCP42100, it was initially decided to avoid the use of the SPI bus. This was since there are enough extra pins for MOSI, CLK, and CS#, and there was a desire to avoid any potential conflict with the external ICSP programmer when first loading the Arduino bootloader. This was accomplished by bit banging.

Communicating to the MCP42100 via Arduino SPI.h

In this example, due to space constraints on the PCB, it was decided to use the SPI.h library included with the Arduino IDE for communicating with SPI devices. The benefit of this approach is that the library utilises the internal SPI core of the microcontroller speeding up communication to the digipot as the SPI core will autonomously send any bytes in its buffer.

To help keep the Arduino sketch clean and for future extensibility, the digipot code was collated into a library.

Setting up the Arduino SPI.h, before use. SPI.setBitOrder(MSBFIRST) should be set, which indicates that the most significant bit of the byte should be sent first. LSBFIRST replaces MSBFIRST if the least significant bit is required to be sent first in some SPI design.

Next the mode of the SPI should be set, which indicates the clock polarity and the clock phase the micro-controller should account for when communicating with the slave over SPI. Clock Polarity is what state the CLK should be when idle. Clock Phase is whether Data is latched on the rising edge or falling edge of CLK.

| | Clock Polarity | Clock Phase |
|---|---|---|
| \|MODE | (CPOL) | (CPHA) |
| SPI_MODE0 | 0 | 0 |
| SPI_MODE1 | 0 | 1 |
| SPI_MODE2 | 1 | 0 |
| SPI_MODE3 | 1 | 1 |

Note:
CPOL: 0 = ‘low idle’; 1 = ‘high idle’
CPHA: 0 = ‘rising edge’; 1 = ‘falling edge’

Reference Source: [34]

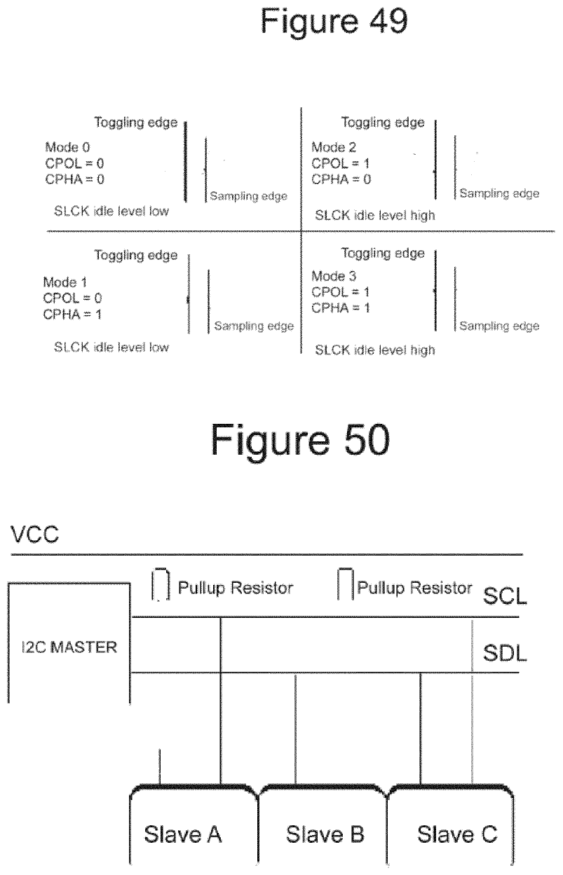

Referring to timing diagram in FIG. 47 and comparing against FIG. 49, it would be best to select positive Clock Polarity and the positive clock edge for Clock Phase:

SPI.setDataMode(SPI MODE0); Sending a byte is conducted by:

SPI.transfer(byte); // Where byte is the byte to be sent to slave

FIG. 50 shows the typical wiring of an I2C device. Note that there is a mandatory need for pullup resistors for SCL and SDL.

I2C (Inter Circuit Communication)

I2C is a proprietary peripheral bus by Philips and the standard consist of two lines—one clock and one data. The advantage of this setup is that the data-line is bidirectional, clocked and bussed. The synchronous nature of the standard means the complexity of the device I2C handler is simplified compared to UART/USART. Clocked data is not dependent on timing and thus require less logic. The bidirectionally and the lack of chip select compared to SPI means decreased PCB complexity, due to reduced numbers of wires to route. There is also an advantage of wide support and selections of I2C devices in the market, making this almost as simple as “plug and play” in engineering terms.

ADXL345 I2C Communication

The posture sensing device uses I2C to access the I2C accelerometer ADXL345. A library based on the Wire.h Arduino standard library was created, which was helpful in the ability to read from two accelerometers.

Key points in ADXL345.cpp library:

Wire.beginTransmission(i2cAddress) Setups a connection to I2C address. The I2C address would refer to the address of the accelerometer.

Wire.endTransmission( ) Tells Arduino to end the connection, and also shows what variables its at.

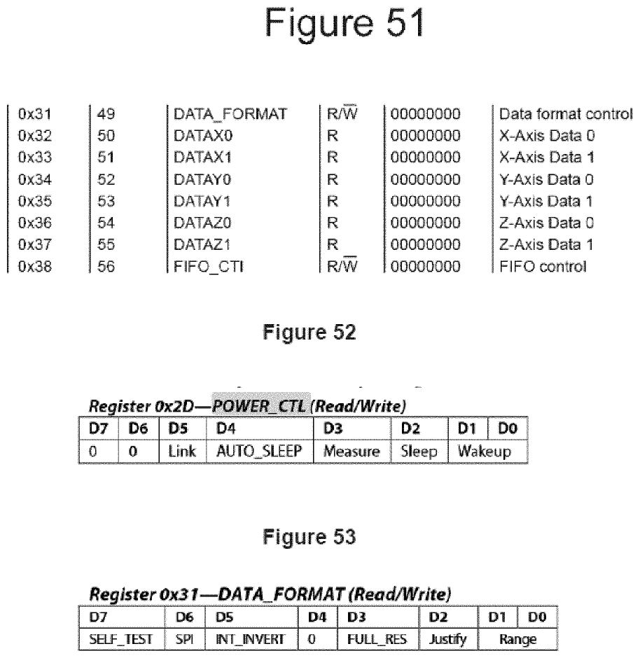

writeToReg(DATA FORMAT, 0×01) Set range to—+4 g. Refer to FIG. 51 for the corresponding bit position, and it's meaning.

writeToReg(POWER CTL, 0×08) Turns on measure bit Setting the measure bit in POWER CTL to 1 to enable measure mode “A setting of 0 means standby mode”, “1 means measurement mode”. ADXL345 is on standby mode by default. Refer to FIG. 52 for the corresponding bit position, and it's meaning.

readFromReg(byte(0×32), 6, accBuff)Set Reg Pointer to 0×32, and read 6 bytes to Byte array at accBuff (Aka: pointer to accBuff[0]). It possible to get all 3 integers values of x, y, z from one call, since the results Registers in FIG. 51 shows that retrieving bytes sequentially between 0×32 to 0×37 will reach all Data 1 and Data 0 results register for all 3 axis.

FIG. 52 shows ADXL345 POWER CTL register from

FIG. 53 shows the ADXL345 DATA FORMAT register from

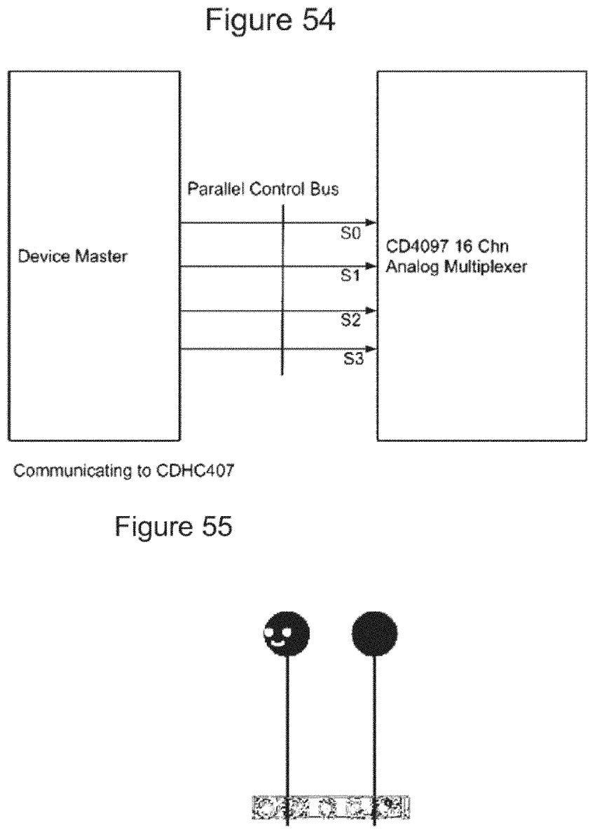

FIG. 54 shows S0 to S3 used as multiplexer channel selection.

Parallel Signalling

Parallel signalling is used for controlling the CD4097 16 channel analog multiplexer used in the final PCB. A 4 bit signal is sent along the control lines S0,S1,S2,S3 as seen in FIG. 54, where S0 is the least significant byte (LSB), and S3 is the most significant byte (MSB). With 4 control lines, 16 channels can be controlled. Changes in control line, leads to near instantaneous changes in multiplexer channels.

The code to control the multiplexer—this function breaks a channel selection value into its corresponding bits to switch the control line of the multiplexer.

Phone Application

The example involves creating a consumer product. In order to decrease costs while increasing adoption, it is important to have the device information easily displayed to the user, on an everyday smartphone instead of a dedicated display device.

FIG. 55 shows the Wireframe draft art of the User Interface for the PhoneGap Application. Left: Front-back flex. Right. Side to side flex

The smartohone application will eventually cover both dominant smartphone ecosystems, Android and iOS. The ecosystem chosen for development was Android, mainly due to no monetary barriers to entry. iOS requires enrolling in the MFi program to use Bluetooth 2.0.

Mobile Development

1. Connect to processing unit via Bluetooth

2. Reconstruct and display a spine curve from sensor data

3. Save the user's optimal position

4. Provide both visual and haptic feedback when spine is not in an optimal position

The following development tools were used—

Native Android SDK uses Java has a steep learning curve, but has the most features and flexibility.

PhoneGap (Apache Cordova) is by Adobe, a cross platform development tool using HTML5 and JavaScript. Bluetooth plugins are available, including device specific features such as Android hardware button support and NFC.

Icenium (Apache Cordova) is also a variant built upon Cordova. Icenium is compat-ible with PhoneGap and its plugins.

Unity's cross platform 3D engine is commonly used for game development. There is no preexisting Bluetooth library.

Corona Labs SDK is cross platform that uses LUA on top of C++ and OpenGL. Blue-tooth support is hazy.

Appcelerator Titanium SDK is an independent HTML and JavaScript based platform. The Bluetooth serial plugin is developed by a third party, and requires licens-ing in the form of seats.

Selection criteria in order of importance—

1. Bluetooth serial support

2. Ease of use, but flexible with good documentation

3. Cost to entry

4 Programming language

| | Bluetooth | | | |
|---|---|---|---|---|
| | (serial) | Documenta- | Programming | Cost to |
| | support | tion | language | develop |
| Native | Yes | 3 | Java | Android Free |
| PhoneGap | Yes | 1 | HTML/JS | Free |
| Icenium | Yes | 3 | HTML/JS | Free |
| Unity | No | 2 | C#/C++ | Free |
| Corona | No | 2 | LUA | Free |
| Labs SDK | | | | |
| Appcelerator | Yes | 2 | JS/XML | Free + |
| Titanium SDK | | | | Bluetooth |
| | | | | serial |
| | | | | licensing |

PhoneGap

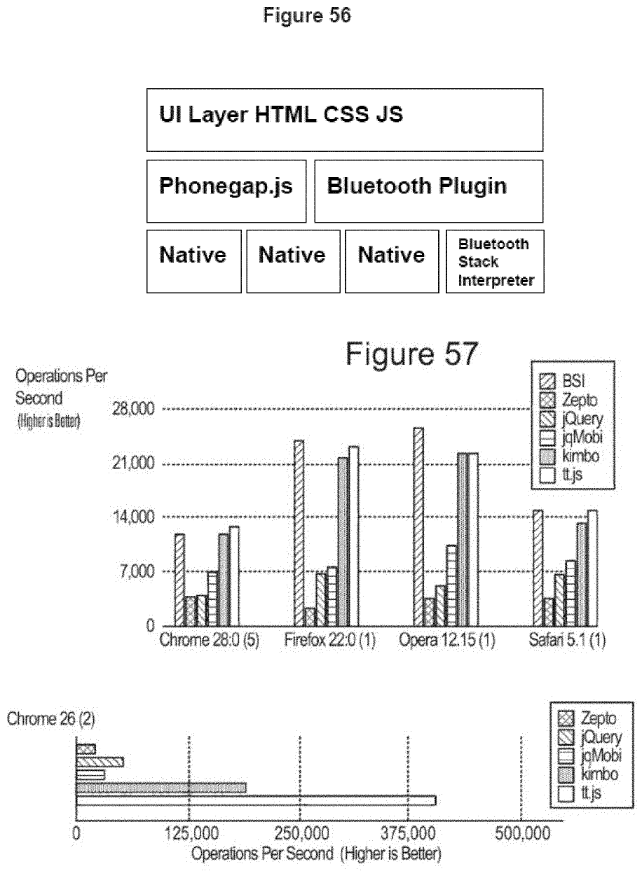

PhoneGap was chosen in this example as the development platform for prototyping. Post prototyping the phone apps will be built in their native platforms eg. iOS and Android. The main reasons for PhoneGap were the amount of accessible documentation and existing support for the Bluetooth serial profile. HTML and JavaScript are both very powerful visual languages despite being less syntax strict. Through the increase in web adoption, JavaScript engines are now comparable in speed compared to native code. FIG. 56 shows The PhoneGap architecture.

PhoneGap consists of phonegap.js, a JavaScript library that acts as the interpreter between the application's JavaScript and native OS Java. Any functionality beyond displaying the application such as hardware button support or Bluetooth is done via PhoneGap plugins.

Smartphone Application

The entire application is contained within one HTML file, with references to JavaScript and styling libraries. Because the application is a web page, all manipulation within the HTML Document Object Model (DOM) must be done through JavaScript. The application uses elements from the latest version of HTML5 and CSS3.

Manipulating the DOM

By using a JavaScript framework like jQuery for DOM manipulation, it is possible to ‘chain’ queries together, by running multiple queries with a single statement, allowing more functionality with less lines of code.

${element-type}.selectingElementByIdentifier( ).action1( ).followedByAction2( ):

Most of the functions are English-named which along with chaining, makes self documentation straightforward. Staying true to the cross platform philosophy, frameworks make OS compatibility easier to manage.

The smartphone application makes use of a jQuery syntax compatible library called tt.js, a high speed implementation of selector based queries designed specifically for mobile devices. The decision was based on the performance of the most commonly used jQ queries within the application, html and addClass functions. By using the jQuery syntax, any compatible library can be used as a direct drop-in replacement, such as the Intel App Framework during Endeavour (major sponsor).

The tt.js library consists of two sections, TTWorker and tt.object. Both are used within the application.

TTWorker is involved in selecting elements and classes, setting visual styles and DOM manipulation.

tt.object processes all the non-visual functions, including parsing JSON, AJAX loading and array manipulations.

FIG. 57 show the Benchmark of popular jQuery syntax compatible libraries for .html and .class functions. Higher bars are better.

Canvas Element

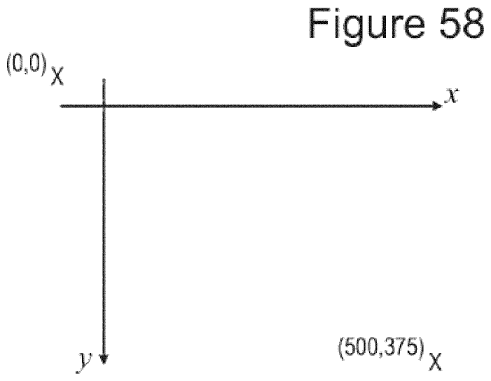

FIG. 58 shows a canvas element. Measurement unit is in pixels. The origin, (0,0) is on the top left. The canvas is 500×375 pixels. The bottom right position is (500, 375). The canvas is a graphical element container that allows manipulation through JavaScript using the HTML5 canvas API. A canvas context object is required, either 2d or webgl (3D) which contains the methods and and properties required to render graphics. Each time the canvas is resized, the contents are cleared. Animations will require clearing of the canvas and redrawing on each new frame as there is no stored memory feature of existing graphics or lines present.

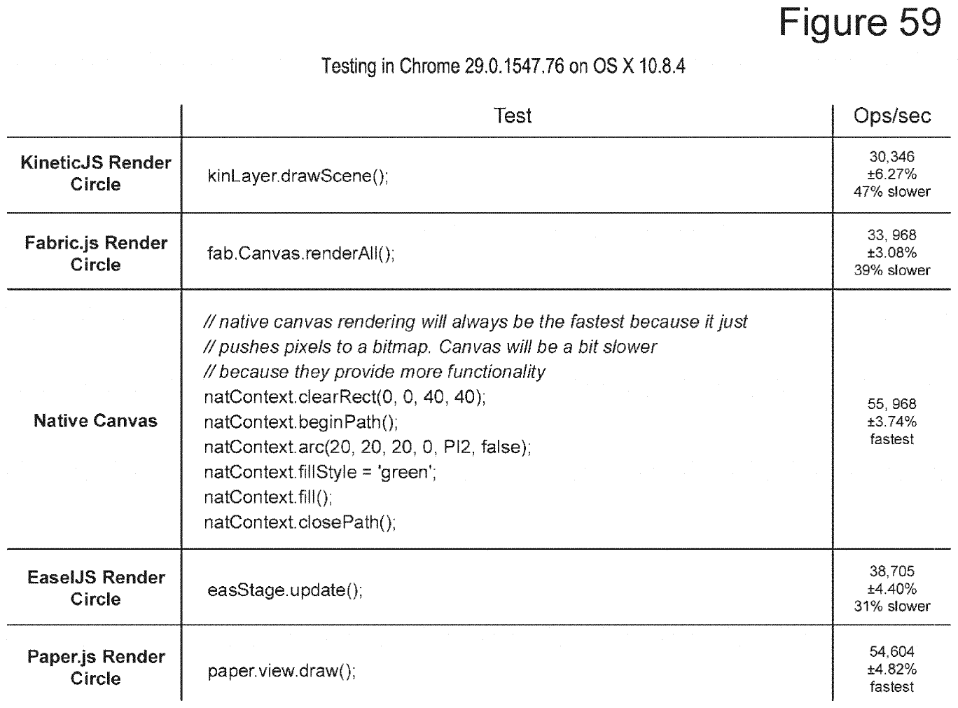

FIG. 59 shows a Comparison of javascript canvas libraries. While native JavaScript can be used to control the canvas element and DOM, there are readily available canvas libraries that abstract difficult functions to make the canvas more easier to manipulate. In native JavaScript, plotting a line would involve a strict syntax of x and y coordinates, [40, 20], [20, 52] . . . compared to the use of libraries which can take the data as objects or nested arrays.

PaperJS Canvas Library

There is a large range of canvas JavaScript libraries available. While JavaScript libraries provide flexibility for the developer, this may sometimes come at the cost of performance. jsperf.com was used to compare the performance of different libraries. While each library has it's own implementation of a specific piece of code, it can provide a rough estimate.

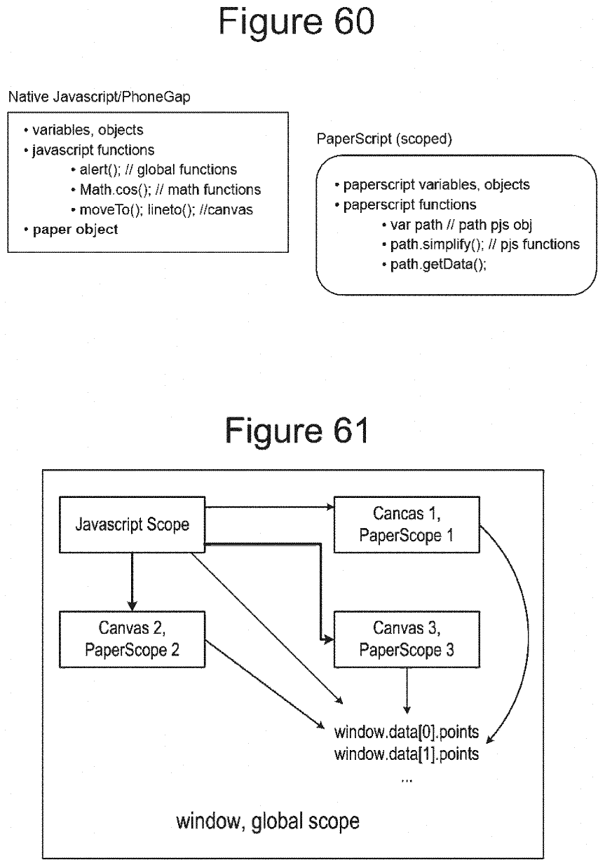

The library PaperJS performed consistently better than the others, with speeds comparable to native speed. Based on the popular Adobe Illustrator plugin Scriptographer, it has a very extensive function library with detailed documentation on the example website. As a bonus, it also has powerful features such as simplifying points to save on computing power. The tradeoffs are the library is relatively new but is under active development; some basic functions such as on demand animations are yet to be developed. The use of PaperScope soaping within JavaScript which can be seen as both advantage (for experts) but disadvantage for newer developers. FIG. 60 shows Javascript and PaperScript scoping.

Each individual canvas element is assigned its own PaperScope. By having a scoped PaperScope with PaperJS, it does not pollute the global public narnespace with variables, and it also means different PaperScript code can be run simultaneously without conflicting one another, which is particularly useful with multiple canvases or parallel manipulations involving similar variable names.

The scoping feature is a difficult concept. As PhoneGap uses JavaScript, it cannot directly access variables in the PaperScope. In order to use PaperScript with general JavaScript functions (or JavaScript libraries like jQuery), the current PaperScope present in the global namespace/javascript space before the features of PaperJS can be used.

An approach is storing each PaperScope as a JavaScript object, such as mypaper[0], mypaper and switching, installing each PaperScope into global namespace before manipulating each canvas element.

The code by Zack Grossbart was posted in the PaperJS Google group. The loader.js file is a function that automates the switching of multiple canvases easily, keeping the while also keeping each paperscript in separate .pjs files for neatness.

loader.js

This code's function checks if the external PaperScript .pjs file and canvas is valid. If both are valid and exist, both are loaded through AJAX and attached to the selected canvas in focus. The code within the selected .pjs file is then evaluated.

Javascript and Paperscript Interoperability

By default, PaperJS cannot work natively with JavaScript variables due to the scoped structure. The Javascript interoperability reference page on PaperJS' website has still yet to be completed by the developer. In order for PaperJS to work with native JavaScript, the variables must be placed into the global namespace suggested by PaperJS developer Jurg Lehni as an interm solution. The variables must be assigned into the global namespace, ‘window’. Not only does this expose a set of PaperScript with JavaScript, but also allows sharing of data between canvases/PaperScopes by having a central data store without passing data via the loader.js function. FIG. 61 shows the JavaScript, PaperScope and window global variables (data store).

Touch Events

Prior to the smartphone, the HTML and Javascript were designed for the desktop computer with a mouse cursor. Java Script only supports clicks, not taps. On a smartphone, this causes a delay in response and/or require a double tap for each button. By using a touch framework, tap and gesture responses can be recognised within the web page application at native speed for the end user. Hammer.js, a jQuery syntax friendly gesture framework is used. Although not part of the jQuery set, chaining can be completed in a similar syntax style

Bluetooth.Serial PhoneGap Plugin

The application makes use of the Bluetooth.Serial plugin developed by Don Coleman. The plugin acts as the interpreter with the default Android Bluetooth stack, Bluedroid. Bluetooth commands are sent via JavaScript and converted into native commands to the Bluetooth radio on the smartphone. Android versions from 2.0 to 4+ are supported. The application makes use of the following pieces of code

bluetoothSerial.subscribe

This instantiates a long running callback to run in the background, which triggers when any new data is received by the processing unit. The subscribe function manages bluetoothSerial.readUntil and the read/write buffer.

bluetoothSerial.write

Sends text via Bluetooth. A success callback is called if successfully sent.

bluetoothSerial.list

Scans the area for previously paired Bluetooth devices and returns data in JSON format. This data is subsequenty processed into a list for the user to select

bluetoothSerial.connect/disconnect

Allows connection/disconnection with the selected device MAC address.

Application Functions

Due to the limitations of scope with PaperJS, there are 4 canvas elements split by direction (front and side) to allow for simultaneous canvas drawing and manipulations, eg: updating one curve/canvas will not mean redrawing the others. As each curve is updated with new data, PaperJS redraws the entire canvas. This is processed on the fly at a high frequency.

The app has the option of calibrating to the optimal spine of the end user, and detect any deviations from this position. The optimal posture reference is updated only once or twice each time you use the application, thus there is no need to continuously redraw with each data update, reducing the amount of processing by half for each direction. The optimal posture reference canvases are layered directly under the data curve to give the appearance of a single canvas element.

On document ready

This is the analogous to onDOMReady with jOuery. The following section of setup queries is run once the phone application has loaded in the device. This sets up and binds the hardware keys, the bluetoothSerial.subscribe callback and Hammer.js queries to detect touches.

Converting Data to JSON

Data sent by the processing unit is a text string in JSON format. The string is parsed into JSON format once new data is received. This initiates canvas manipulations.

Calculating Curve Positions

Curve position calculations are completed by trigonometry from the bottom position. The code calculates the amount of data points received in the data object and estimates the length of the spine. The curve is simplified using PaperJS inbuilt smoothing functions, and the resultant spine curve is displayed within the canvas.

Haptic Feedback

Signals are sent to the processing unit via a designated single character string. The code can choose from a varied selection of pulses and vibration power.

Calculating the Deviation from Optimal Posture

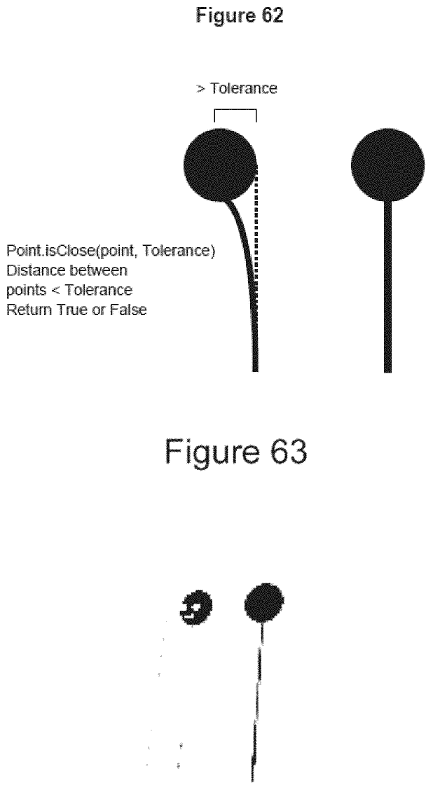

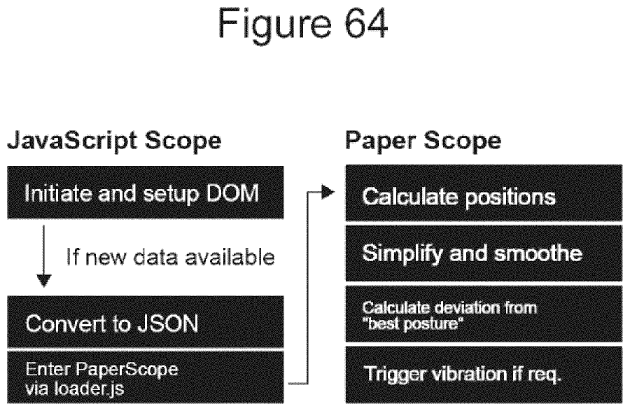

FIG. 62 shows calculating the deviation from user-saved best posture position

FIG. 63 shows a 3D representation of the mobile application. Separate canvas for each curve. Each time new data is being sent, before the curve is being displayed, the PaperJS code runs a comparison check to see if certain points along the body fall within a tolerance threshold, measured by distance. If the distance is greater than the tolerance, it will trigger a haptic feedback signal within the processing unit via Bluetooth followed by a visual colour change of the figure. Multiple points can be used as a comparison once suitable threshold levels are obtained from a medical professional. This profile can be saved within the app and additional reference profiles can be loaded on the fly, which would make it possible for specific postures, such as for sports or Yoga.

Performance

Rounding Sub-Pixels

HTML canvas supports sub-pixel rendering/anti-aliasing if the supplied data points are in the form of floating numbers. This causes performance issues in various browsers such as IOS and Mac platforms. Since our formula to calculate points sometimes gives non-integer values, rounding may be an necessary option to increase speed.

Options involve using built in Math.floor rounding or hacks. Hacks seem to perform faster, e.g. adding 0.5 to the number, and truncate to zero decimal point, with bit-wise shift.

rounded=˜˜(0.5+somenum); http://jsperf.com/math-round-vs-hack/3

Using Lookup Tables instead of JavaScript.Math Functions

Calculating the curve points using are computationally expensive. Currently, inbuilt JavaScript Math functions are used each time the curve is updated. The idea of using a lookup table in replacement for sine and cosine functions. If using Math functions as the data is given in degrees, besides the trigonometric calculation we also need to convert degrees value to radians, with respect to floating Math.PI (or cached rounded value).

var cosLUT=[preprecalculated values for degrees] cosLookup(angle): takes angle as nth position in cosLUT return value at angle/nth position

Input of the function is populating an array (array nth element=degrees lookup). The function would need to take negative degrees into consideration as well, and the extra conditional statement is needed. This produced varying results, http://jsperf.com/ testing-lut-neg-and-trig-functions.

Originally, the lookup table LUT technique was significantly faster than using Math functions. Testing with Chrome 30.0 results in a LUT advantage more consistent with OS platforms. On the Samsung Galaxy S2, the Math function was faster than LUT by factor of 18 (18M Op/sec). A separate test with rounded LUT values to 5 dp showed a consistent increase compared one with more decimal places. This approach has been placed on hold until more consistent testing can be completed.

For Loop Efficiency

A loop calculating the curve points from acquired data is run each time a new set of data is received by the MCU via bluetooth. This high frequency piece of code therefore needs to be very fast in order to save computing power. By increasing the number of calcuiations/sec, the speed of curve plotting can be significantly improved and load reduced.

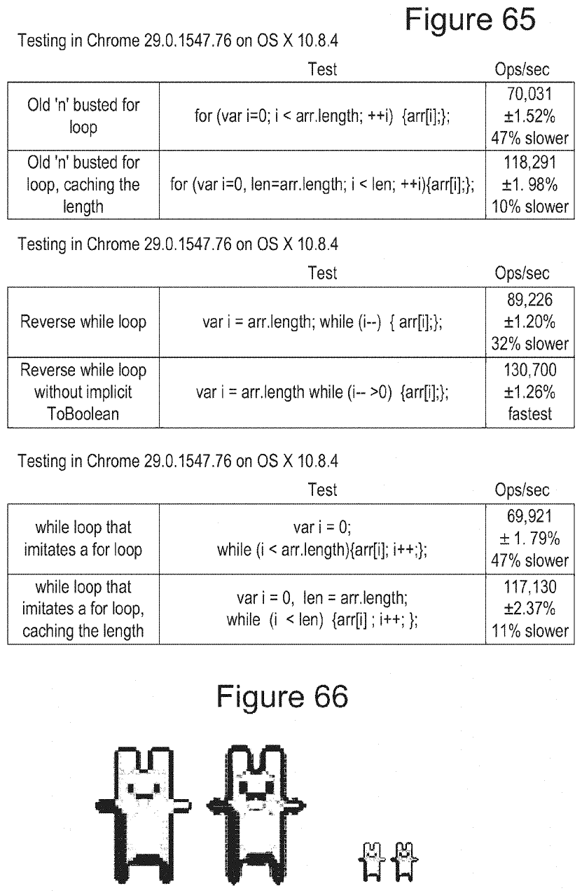

FIG. 65 shows a jsperf.com benchmark with many loop implementations is available. Looping performance benchmark.

The current function was in the format of old n busted. Without rewriting an initial parameter caching the length the for loop runs to, we can increase operations/second by a factor of 1.5. The while implementations are negative counter (comparison against 0, not a variable like len), which may reduce computing cycles. While our curve point loop is different, this small addition allows us to address this without drastic alterations to current code. Note: this varies depending on which browser is used as not all JavaScript engines are created equal. In addition, i++ and ++i incrementing work the same way.

FIG. 66 shows—Left: No subpixel rendering (aliased), Right: Antialiasing is used to smooth the sprite as the origin point is not a set integer.)

Certain implementations of the WebView across both iOS and Android support floating numbers for points on the canvas. The WebView renders these across multiple pixels nearest in the form of anti-aliasing. This results in extra computations to smooth out the graphics. In our case, most of the calculations are in decimal places, thus it this is also another area to focus on to speed up the application.

Bluetooth Error Detection

Whilst there was initially bluetooth character dropping issue, we considered whether to add a cyclic redundancy check suffixed to the end of the JSON string to check if the JSON string itself was valid. The mobile application would encode the JSON string and compare it with the CRC attached. The data would only be processed by the curve updater function if passing this check. Any corruption to any of the two would result in the string being dropped. This is considered a safe-side failure. The dangerous-side failure is both the checksum and data are corrupted such that they are both consistent, but its probability can be minimized by increasing the checksum length, at a cost of longer strings may trigger higher probability. This approach has been discarded since the Bluetooth plugin has been updated to rule out this error altogether.

Measurement Rig Design

To help speed measurements, a manual rig and an automated rig was created. Please refer to Appendix A.3 for the manual rig.

Automated Rig

For the automated rig, it consist of a foam base, with a 6V servo motor hooked up to a Sparkfun 3.3V pro micro board. Voltage reading is done automatically via the Sparkfun 3.3V pro micro board. For resistance readings, many hour was taken up manually reading a standard multimeter.

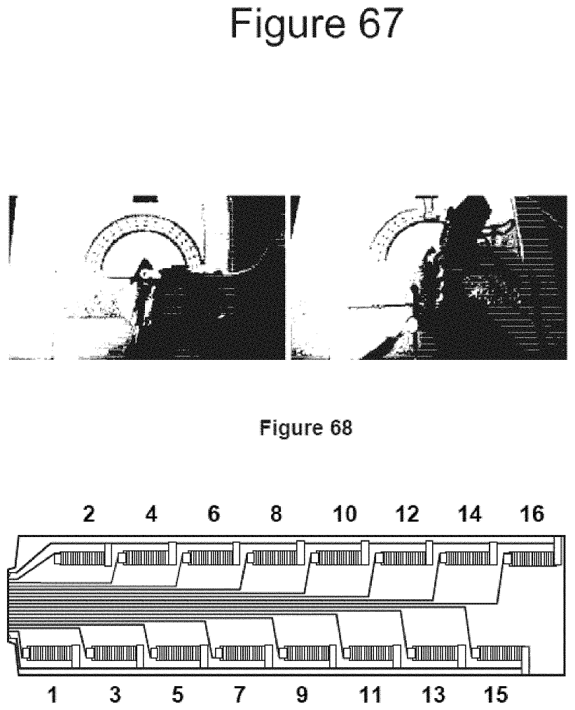

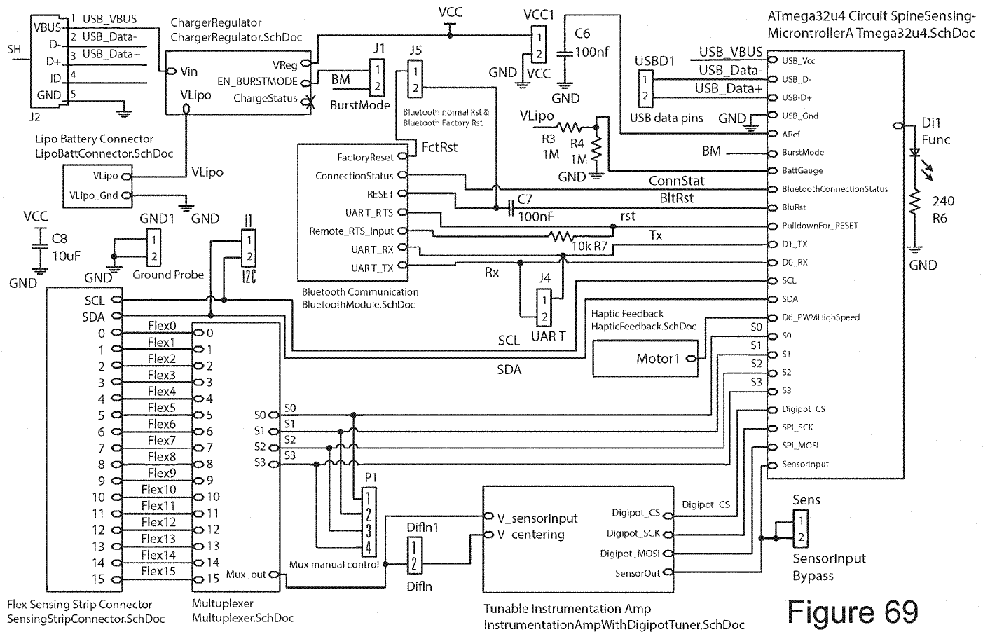

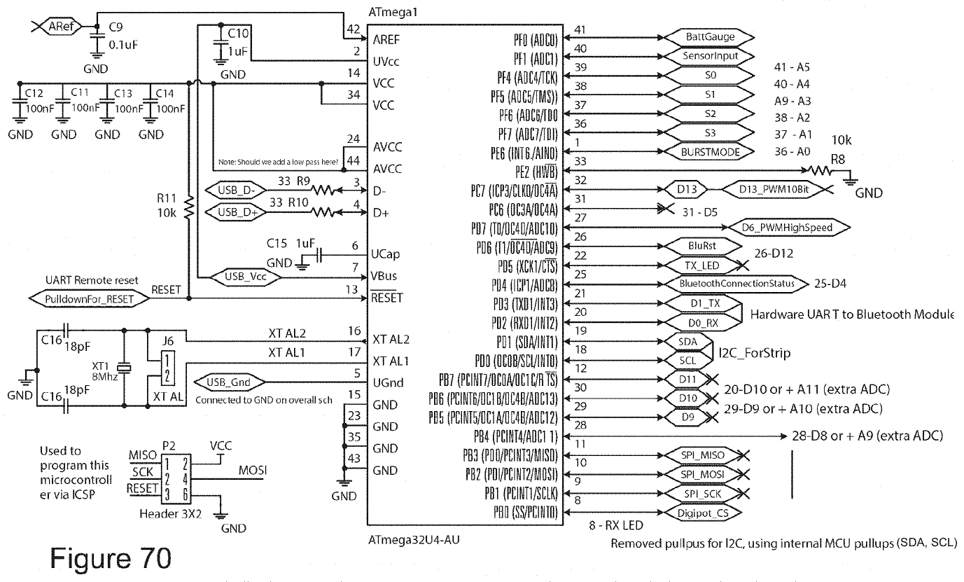

FIG. 67 shows firstly the automated rig testing a flex strip and secondly the automated rig testing an optical break flex sensor.

In terms of servo accuracy, it is dependent on the angle of the servo motor. It is most accurate at zero flex or position 90 (0 to 180) or 0 (−90 to 90), can expect a tolerance of +−1 degree. However the tolerance diverges up to +−8 degrees when moving towards either side of the servo motor.

Comments on Testing for this Example

For accelerometers, the side to side motion worked as expected, however this is being interefered with by results from forward and backwards sensing.

As for the flex sensors the output has good DC performance, but the movement is jittery. This can be solved by using averages such as an exponential moving average filter. Unfortunately there is no success with implementing an EMA filter onto the micro controller due to implementation problems from lack of micro controller ram.

Zeroing calibration worked well which means that the rest flex was able to be reached consistently at all trials. The magnitude of the flex sensor response also appears to be consistent.

In terms of the phone app, the app is able to receive and parse JSON commands to display a curve. It was also able to ‘software’ calibrate to a nominal spine curve, so that the app knows when to trigger an alarm. This alarm could either be displayed as an indicator in the visual display, or as vibrational notification on the posture sensing device.

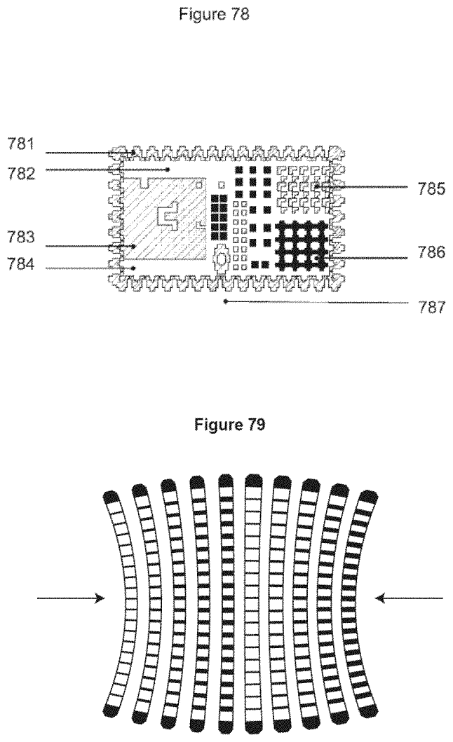

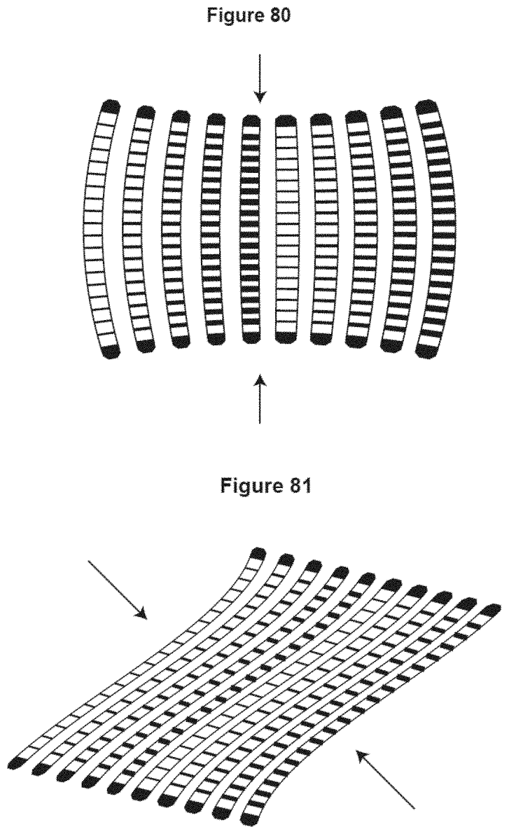

FIG. 78 depicts an example strip according to the invention showing external contact points 781, dielectric (top layer) 782, silver conductive ink 783 and 785, substrate 784, carbon resistive ink 786 and body adhesive 787 on the reverse side. FIG. 79 depicts an example strip according to the invention showing horizontal displacement. FIG. 80 depicts an example strip according to the invention showing diagonal displacement. FIG. 81 depicts an example strip according to the invention showing vertical displacement.

In some preferred embodiments the thickness of the strip sensor substrate (for example shown also in FIG. 20) is less than 100 microns, and preferably 60 to 80 microns and in some preferred embodiments it is about 75 microns. In some embodiments, a substrate such as CT3 (Autostat) may be used. However, in more preferred embodiments additional multi-dimensional movement is allowed.

In some preferred embodiments, there is provided a sensor strip comprising:
1. Multi-dimensional surface;
2. Carbon resistive Ink;
3. Conductive Ink;
4. Dielectric Cover;
5. Body Adhesive to use as a sensor on the human body; and
6. Removable Adhesive Backing.

FIGS. 78-81 show the static position (for example before application) and then the combined horizontal displacement, diagonal and vertical displacement of the sensor strip. These movements can also be considered in combination providing a multi-dimensional capability similar to the human skin or an item of clothing. Directions indicated mean a 3-dimensional displacement or stretching within the limits of the substrate before tearing occurs of the substrate or printed inks (which would render the sensor inaccurate).

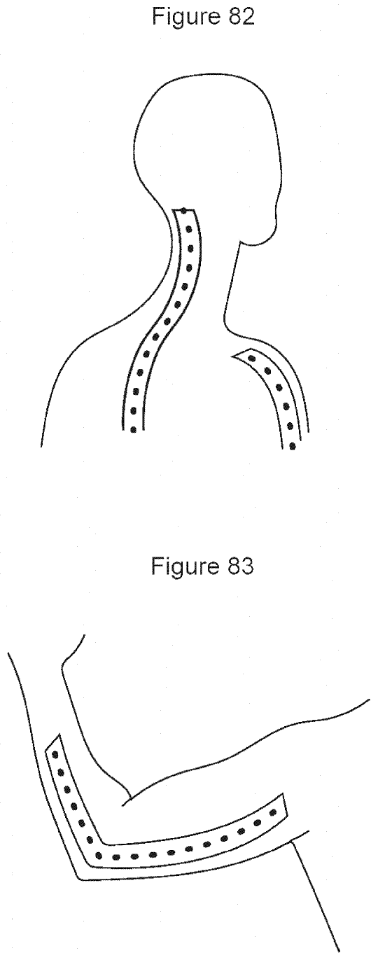

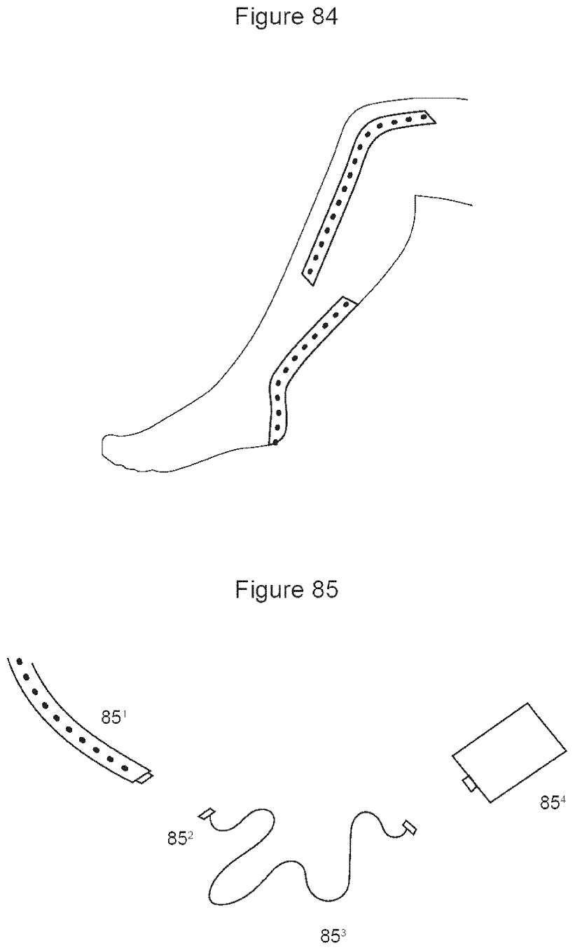

FIGS. 82-84 show examples embodiments in which a strip of the invention is utilised with different body portions. Whilst strips of the invention can be used with any part of the body, particularly suitable body portions comprise: parts of the back, Shoulders and Neck but also the elbow, knee, ankle. Body portions that experience RSI (repetitive strain injury) and joints in the body are all possible extensions for the technology. Strips according to the invention may be modified to fit particular body portions and the mobile application available to register and provide a response to unique body portions. In some embodiments, the invention comprises measurement and/or tracking of one or more body portions, which may occur simultaneously.

FIG. 85 depicts use of one embodiment of the invention comprising direct connection between a strip and a transmitter. Benefits of such an embodiment include for example allowing it to be stored for ready access, such as in the pocket of the individual wearing the strip.

Example Strip Test

The following test procedure was performed and generated the data set out below.

1. resistive and conductive ink on adhesive strips were attached to the spine of a volunteer;

2. this strip is connected to the microprocessor transmission device;

3. the transmission device connects using bluetooth to a mobile phone;

4. the transmission device sends raw resistance data in real time for each of up to 16 sensors on the strip to the mobile phone;

5. the mobile phone runs an application which is calibrated first at a neutral spine position for the person;

6. the mobile phone app collates the real-time resistive data and provides a visual demonstration and alerts the user if ‘out of posture’; and

7. the mobile phone app logs the data and therefore history of the data provided by the strip via the transmission device.

TABLE 1
timing of test events

| Time (mm:ss) | Event |
|---|---|
| 00:00 | start testing |
| 00:15 | move back to lightly bend |
| 00:30 | return to straight |
| 00:45 | move back to normally bend |
| 01:00 | return to straight |
| 01:15 | move back to extremely bend |
| 01:30 | return to straight |
| 01:45 | end testing |

TABLE 2
resistance values (ohms) at each second for channels 1-8

| Time | Channel | Channel | Channel | Channel | Channel | Channel | Channel | Channel |
|---|---|---|---|---|---|---|---|---|
| elapsed | 1 | 2 | 3 | 4 | 5 | 6 | 7 | 8 |
| 1 | 936 | 958 | 1023 | 1035 | 1041 | 1058 | 1061 | 1051 |
| 2 | 937 | 958 | 1024 | 1035 | 1042 | 1058 | 1061 | 1051 |
| 3 | 936 | 958 | 1023 | 1035 | 1041 | 1059 | 1061 | 1049 |
| 4 | 937 | 958 | 1024 | 1035 | 1041 | 1057 | 1061 | 1049 |
| 5 | 937 | 958 | 1024 | 1035 | 1040 | 1058 | 1062 | 1049 |
| 6 | 935 | 958 | 1024 | 1035 | 1041 | 1057 | 1062 | 1049 |
| 7 | 937 | 958 | 1024 | 1035 | 1041 | 1057 | 1060 | 1049 |
| 8 | 936 | 958 | 1024 | 1035 | 1040 | 1058 | 1061 | 1049 |
| 9 | 935 | 959 | 1024 | 1035 | 1041 | 1058 | 1062 | 1049 |
| 10 | 936 | 958 | 1024 | 1035 | 1040 | 1059 | 1061 | 1051 |
| 11 | 935 | 958 | 1024 | 1034 | 1042 | 1057 | 1061 | 1049 |
| 12 | 936 | 958 | 1023 | 1034 | 1042 | 1058 | 1061 | 1049 |
| 13 | 936 | 957 | 1021 | 1034 | 1040 | 1058 | 1061 | 1049 |
| 14 | 936 | 958 | 1023 | 1034 | 1041 | 1057 | 1061 | 1049 |
| 15 | 936 | 958 | 1024 | 1037 | 1041 | 1058 | 1061 | 1051 |
| 16 | 936 | 959 | 1023 | 1037 | 1041 | 1055 | 1061 | 1051 |
| 17 | 936 | 962 | 1023 | 1039 | 1044 | 1055 | 1062 | 1053 |
| 18 | 938 | 962 | 1023 | 1039 | 1044 | 1057 | 1064 | 1054 |
| 19 | 938 | 962 | 1024 | 1039 | 1044 | 1055 | 1062 | 1053 |
| 20 | 938 | 962 | 1024 | 1039 | 1045 | 1055 | 1062 | 1053 |
| 21 | 938 | 962 | 1024 | 1039 | 1044 | 1057 | 1064 | 1054 |
| 22 | 938 | 963 | 1024 | 1039 | 1044 | 1057 | 1064 | 1053 |
| 23 | 938 | 962 | 1024 | 1039 | 1045 | 1057 | 1064 | 1054 |
| 24 | 939 | 960 | 1024 | 1039 | 1045 | 1055 | 1064 | 1054 |
| 25 | 938 | 962 | 1024 | 1039 | 1045 | 1055 | 1064 | 1054 |
| 26 | 938 | 963 | 1024 | 1039 | 1045 | 1055 | 1062 | 1054 |
| 27 | 939 | 960 | 1024 | 1040 | 1045 | 1055 | 1064 | 1054 |
| 28 | 938 | 962 | 1025 | 1039 | 1045 | 1057 | 1064 | 1054 |
| 29 | 939 | 962 | 1024 | 1039 | 1045 | 1057 | 1064 | 1054 |
| 30 | 938 | 962 | 1025 | 1038 | 1045 | 1055 | 1064 | 1054 |
| 31 | 938 | 962 | 1024 | 1039 | 1045 | 1055 | 1064 | 1054 |
| 32 | 938 | 960 | 1024 | 1039 | 1045 | 1059 | 1064 | 1053 |
| 33 | 937 | 959 | 1025 | 1039 | 1042 | 1060 | 1062 | 1052 |
| 34 | 937 | 960 | 1025 | 1038 | 1044 | 1060 | 1062 | 1052 |
| 35 | 935 | 959 | 1024 | 1038 | 1042 | 1060 | 1061 | 1052 |
| 36 | 936 | 960 | 1024 | 1038 | 1042 | 1060 | 1061 | 1052 |
| 37 | 937 | 960 | 1024 | 1038 | 1042 | 1059 | 1062 | 1052 |
| 38 | 937 | 960 | 1024 | 1037 | 1042 | 1059 | 1061 | 1052 |
| 39 | 936 | 960 | 1024 | 1038 | 1042 | 1059 | 1062 | 1052 |
| 40 | 937 | 960 | 1024 | 1038 | 1042 | 1059 | 1062 | 1052 |
| 41 | 937 | 960 | 1025 | 1038 | 1044 | 1059 | 1061 | 1052 |
| 42 | 937 | 960 | 1024 | 1038 | 1042 | 1059 | 1062 | 1052 |
| 43 | 937 | 962 | 1024 | 1037 | 1042 | 1059 | 1061 | 1052 |
| 44 | 937 | 960 | 1025 | 1038 | 1042 | 1060 | 1062 | 1052 |
| 45 | 937 | 960 | 1024 | 1037 | 1042 | 1060 | 1061 | 1052 |
| 46 | 937 | 960 | 1024 | 1037 | 1042 | 1060 | 1061 | 1052 |
| 47 | 937 | 960 | 1024 | 1038 | 1042 | 1060 | 1061 | 1052 |
| 48 | 937 | 970 | 1024 | 1042 | 1042 | 1059 | 1061 | 1057 |
| 49 | 944 | 967 | 1026 | 1041 | 1049 | 1059 | 1067 | 1060 |
| 50 | 942 | 966 | 1027 | 1041 | 1051 | 1059 | 1068 | 1059 |
| 51 | 943 | 967 | 1028 | 1042 | 1051 | 1059 | 1067 | 1059 |
| 52 | 943 | 967 | 1028 | 1042 | 1052 | 1059 | 1068 | 1059 |
| 53 | 944 | 967 | 1028 | 1042 | 1052 | 1058 | 1068 | 1059 |
| 54 | 945 | 967 | 1028 | 1042 | 1053 | 1059 | 1071 | 1059 |
| 55 | 944 | 967 | 1028 | 1041 | 1053 | 1059 | 1069 | 1059 |
| 56 | 943 | 967 | 1028 | 1041 | 1053 | 1059 | 1069 | 1059 |
| 57 | 944 | 967 | 1028 | 1042 | 1053 | 1058 | 1069 | 1060 |
| 58 | 944 | 967 | 1028 | 1041 | 1053 | 1058 | 1069 | 1060 |
| 59 | 945 | 969 | 1030 | 1042 | 1055 | 1058 | 1069 | 1060 |
| 60 | 945 | 966 | 1030 | 1041 | 1054 | 1058 | 1071 | 1058 |
| 61 | 945 | 969 | 1028 | 1041 | 1053 | 1058 | 1069 | 1058 |
| 62 | 946 | 967 | 1030 | 1041 | 1054 | 1058 | 1071 | 1060 |
| 63 | 945 | 964 | 1027 | 1044 | 1053 | 1059 | 1069 | 1058 |
| 64 | 941 | 960 | 1024 | 1041 | 1044 | 1062 | 1064 | 1054 |
| 65 | 937 | 960 | 1025 | 1040 | 1042 | 1062 | 1064 | 1052 |
| 66 | 937 | 959 | 1026 | 1039 | 1042 | 1061 | 1062 | 1052 |
| 67 | 936 | 960 | 1026 | 1039 | 1041 | 1060 | 1064 | 1052 |
| 68 | 936 | 958 | 1026 | 1040 | 1041 | 1059 | 1064 | 1051 |
| 69 | 936 | 960 | 1025 | 1040 | 1042 | 1059 | 1062 | 1051 |
| 70 | 937 | 960 | 1026 | 1040 | 1041 | 1059 | 1062 | 1052 |
| 71 | 937 | 959 | 1026 | 1039 | 1041 | 1060 | 1062 | 1052 |
| 72 | 936 | 960 | 1025 | 1039 | 1042 | 1060 | 1062 | 1053 |
| 73 | 937 | 960 | 1025 | 1040 | 1042 | 1059 | 1062 | 1052 |
| 74 | 936 | 960 | 1025 | 1039 | 1042 | 1059 | 1062 | 1052 |
| 75 | 937 | 960 | 1025 | 1039 | 1042 | 1060 | 1064 | 1052 |
| 76 | 937 | 960 | 1025 | 1039 | 1041 | 1059 | 1062 | 1052 |
| 77 | 937 | 960 | 1025 | 1040 | 1042 | 1059 | 1064 | 1052 |
| 78 | 937 | 960 | 1024 | 1040 | 1041 | 1059 | 1062 | 1052 |
| 79 | 939 | 974 | 1026 | 1046 | 1045 | 1059 | 1065 | 1060 |
| 80 | 946 | 972 | 1035 | 1045 | 1062 | 1058 | 1076 | 1059 |
| 81 | 944 | 971 | 1032 | 1045 | 1060 | 1058 | 1074 | 1058 |
| 82 | 945 | 971 | 1033 | 1044 | 1061 | 1057 | 1075 | 1059 |
| 83 | 945 | 972 | 1034 | 1044 | 1060 | 1057 | 1075 | 1058 |
| 84 | 945 | 971 | 1034 | 1044 | 1060 | 1057 | 1076 | 1059 |
| 85 | 945 | 970 | 1034 | 1044 | 1060 | 1055 | 1078 | 1058 |
| 86 | 946 | 971 | 1034 | 1045 | 1060 | 1057 | 1076 | 1059 |
| 87 | 946 | 973 | 1035 | 1046 | 1061 | 1058 | 1078 | 1060 |
| 88 | 949 | 974 | 1038 | 1047 | 1064 | 1061 | 1080 | 1062 |
| 89 | 950 | 978 | 1039 | 1048 | 1065 | 1062 | 1082 | 1065 |
| 90 | 952 | 976 | 1040 | 1048 | 1066 | 1062 | 1082 | 1062 |
| 91 | 950 | 973 | 1038 | 1046 | 1065 | 1061 | 1082 | 1061 |
| 92 | 949 | 972 | 1037 | 1042 | 1062 | 1058 | 1080 | 1059 |
| 93 | 946 | 967 | 1034 | 1040 | 1060 | 1055 | 1076 | 1055 |
| 94 | 944 | 967 | 1032 | 1038 | 1057 | 1052 | 1073 | 1054 |
| 95 | 943 | 971 | 1030 | 1049 | 1054 | 1065 | 1071 | 1060 |
| 96 | 941 | 966 | 1028 | 1042 | 1047 | 1065 | 1066 | 1055 |
| 97 | 938 | 967 | 1027 | 1042 | 1044 | 1064 | 1065 | 1055 |
| 98 | 938 | 966 | 1028 | 1042 | 1045 | 1062 | 1066 | 1055 |
| 99 | 937 | 967 | 1028 | 1041 | 1044 | 1062 | 1064 | 1055 |
| 100 | 937 | 965 | 1028 | 1042 | 1042 | 1062 | 1064 | 1055 |
| 101 | 937 | 965 | 1028 | 1040 | 1042 | 1062 | 1064 | 1054 |
| 102 | 937 | 965 | 1026 | 1040 | 1042 | 1061 | 1064 | 1054 |
| 103 | 937 | 965 | 1027 | 1042 | 1044 | 1061 | 1064 | 1055 |
| 104 | 937 | 964 | 1027 | 1042 | 1044 | 1061 | 1064 | 1055 |
| 105 | 937 | 964 | 1026 | 1040 | 1042 | 1062 | 1064 | 1055 |
| 106 | 937 | 964 | 1026 | 1040 | 1042 | 1061 | 1062 | 1055 |
| 107 | 936 | 964 | 1026 | 1040 | 1042 | 1061 | 1062 | 1055 |
| 108 | 936 | 963 | 1026 | 1040 | 1042 | 1061 | 1062 | 1053 |
| 109 | 937 | 963 | 1025 | 1040 | 1041 | 1061 | 1064 | 1054 |

TABLE 3
resistance values (ohms) at each second for channels 9-16.

| Time | Channel | Channel | Channel | Channel | Channel | Channel | Channel | Channel |
|---|---|---|---|---|---|---|---|---|
| elapse | 9 | 10 | 11 | 12 | 13 | 14 | 15 | 16 |
| 1 | 1091 | 1079 | 1091 | 1086 | 1094 | 1107 | 1127 | 1120 |
| 2 | 1091 | 1079 | 1091 | 1086 | 1094 | 1106 | 1127 | 1119 |
| 3 | 1091 | 1079 | 1092 | 1085 | 1094 | 1107 | 1127 | 1119 |
| 4 | 1091 | 1078 | 1091 | 1086 | 1094 | 1107 | 1127 | 1119 |
| 5 | 1092 | 1079 | 1092 | 1087 | 1094 | 1108 | 1127 | 1120 |
| 6 | 1092 | 1079 | 1092 | 1087 | 1094 | 1108 | 1127 | 1117 |
| 7 | 1091 | 1078 | 1092 | 1087 | 1094 | 1107 | 1127 | 1117 |
| 8 | 1091 | 1078 | 1092 | 1086 | 1093 | 1107 | 1127 | 1119 |
| 9 | 1092 | 1079 | 1091 | 1087 | 1094 | 1107 | 1127 | 1120 |
| 10 | 1092 | 1079 | 1092 | 1087 | 1094 | 1107 | 1127 | 1120 |
| 11 | 1091 | 1078 | 1092 | 1087 | 1094 | 1107 | 1126 | 1119 |
| 12 | 1091 | 1079 | 1091 | 1086 | 1094 | 1106 | 1127 | 1119 |
| 13 | 1091 | 1079 | 1091 | 1086 | 1094 | 1107 | 1126 | 1119 |
| 14 | 1091 | 1079 | 1091 | 1087 | 1093 | 1107 | 1126 | 1119 |
| 15 | 1091 | 1079 | 1091 | 1088 | 1093 | 1107 | 1127 | 1119 |
| 16 | 1091 | 1079 | 1092 | 1086 | 1094 | 1107 | 1126 | 1119 |
| 17 | 1093 | 1081 | 1089 | 1086 | 1098 | 1108 | 1128 | 1119 |
| 18 | 1093 | 1082 | 1091 | 1086 | 1098 | 1108 | 1128 | 1121 |
| 19 | 1095 | 1081 | 1091 | 1086 | 1100 | 1109 | 1129 | 1121 |
| 20 | 1095 | 1080 | 1089 | 1087 | 1098 | 1108 | 1129 | 1120 |
| 21 | 1094 | 1081 | 1091 | 1086 | 1099 | 1108 | 1129 | 1120 |
| 22 | 1095 | 1082 | 1091 | 1086 | 1100 | 1109 | 1129 | 1120 |
| 23 | 1095 | 1083 | 1091 | 1087 | 1099 | 1109 | 1129 | 1120 |
| 24 | 1095 | 1083 | 1091 | 1086 | 1099 | 1109 | 1132 | 1120 |
| 25 | 1094 | 1081 | 1091 | 1086 | 1099 | 1109 | 1129 | 1120 |
| 26 | 1094 | 1081 | 1089 | 1086 | 1099 | 1109 | 1129 | 1120 |
| 27 | 1094 | 1082 | 1091 | 1087 | 1099 | 1109 | 1129 | 1120 |
| 28 | 1094 | 1083 | 1092 | 1086 | 1099 | 1109 | 1130 | 1120 |
| 29 | 1094 | 1081 | 1091 | 1085 | 1099 | 1109 | 1130 | 1120 |
| 30 | 1096 | 1082 | 1089 | 1085 | 1099 | 1109 | 1132 | 1121 |
| 31 | 1094 | 1082 | 1092 | 1086 | 1100 | 1109 | 1129 | 1120 |
| 32 | 1095 | 1080 | 1091 | 1087 | 1098 | 1109 | 1129 | 1120 |
| 33 | 1094 | 1080 | 1094 | 1089 | 1096 | 1109 | 1129 | 1120 |
| 34 | 1094 | 1081 | 1092 | 1087 | 1096 | 1108 | 1128 | 1121 |
| 35 | 1092 | 1081 | 1092 | 1087 | 1096 | 1108 | 1129 | 1121 |
| 36 | 1094 | 1080 | 1092 | 1088 | 1095 | 1109 | 1129 | 1120 |
| 37 | 1094 | 1080 | 1092 | 1088 | 1095 | 1108 | 1129 | 1120 |
| 38 | 1093 | 1080 | 1092 | 1087 | 1096 | 1108 | 1129 | 1120 |
| 39 | 1093 | 1080 | 1092 | 1087 | 1096 | 1108 | 1128 | 1120 |
| 40 | 1092 | 1080 | 1092 | 1087 | 1096 | 1108 | 1128 | 1121 |
| 41 | 1094 | 1080 | 1092 | 1089 | 1095 | 1108 | 1129 | 1120 |
| 42 | 1093 | 1080 | 1092 | 1087 | 1095 | 1109 | 1128 | 1120 |
| 43 | 1093 | 1080 | 1092 | 1088 | 1096 | 1108 | 1128 | 1120 |
| 44 | 1093 | 1081 | 1091 | 1087 | 1095 | 1108 | 1128 | 1120 |
| 45 | 1092 | 1080 | 1092 | 1088 | 1094 | 1108 | 1128 | 1120 |
| 46 | 1092 | 1080 | 1092 | 1087 | 1094 | 1108 | 1128 | 1120 |
| 47 | 1093 | 1081 | 1092 | 1089 | 1095 | 1108 | 1128 | 1120 |
| 48 | 1093 | 1083 | 1091 | 1086 | 1096 | 1108 | 1128 | 1120 |
| 49 | 1098 | 1085 | 1093 | 1089 | 1103 | 1110 | 1133 | 1124 |
| 50 | 1098 | 1087 | 1093 | 1089 | 1103 | 1112 | 1132 | 1124 |
| 51 | 1098 | 1085 | 1094 | 1089 | 1103 | 1112 | 1133 | 1124 |
| 52 | 1099 | 1087 | 1093 | 1088 | 1103 | 1113 | 1134 | 1124 |
| 53 | 1099 | 1086 | 1093 | 1089 | 1105 | 1113 | 1133 | 1124 |
| 54 | 1099 | 1086 | 1093 | 1088 | 1105 | 1113 | 1133 | 1123 |
| 55 | 1098 | 1086 | 1093 | 1089 | 1105 | 1112 | 1134 | 1123 |
| 56 | 1098 | 1086 | 1093 | 1088 | 1105 | 1112 | 1134 | 1124 |
| 57 | 1099 | 1086 | 1093 | 1089 | 1105 | 1113 | 1133 | 1123 |
| 58 | 1099 | 1087 | 1094 | 1089 | 1105 | 1112 | 1133 | 1124 |
| 59 | 1100 | 1087 | 1093 | 1091 | 1105 | 1112 | 1133 | 1124 |
| 60 | 1099 | 1087 | 1093 | 1089 | 1105 | 1113 | 1134 | 1123 |
| 61 | 1098 | 1086 | 1093 | 1089 | 1105 | 1113 | 1133 | 1123 |
| 62 | 1100 | 1086 | 1093 | 1089 | 1105 | 1113 | 1133 | 1124 |
| 63 | 1098 | 1083 | 1093 | 1088 | 1103 | 1110 | 1133 | 1122 |
| 64 | 1095 | 1080 | 1093 | 1091 | 1098 | 1108 | 1130 | 1121 |
| 65 | 1091 | 1080 | 1093 | 1091 | 1096 | 1108 | 1128 | 1121 |
| 66 | 1091 | 1080 | 1091 | 1091 | 1095 | 1108 | 1129 | 1119 |
| 67 | 1091 | 1080 | 1092 | 1092 | 1095 | 1106 | 1128 | 1121 |
| 68 | 1091 | 1080 | 1091 | 1091 | 1095 | 1106 | 1128 | 1121 |
| 69 | 1089 | 1080 | 1091 | 1091 | 1095 | 1108 | 1128 | 1121 |
| 70 | 1091 | 1080 | 1091 | 1091 | 1095 | 1108 | 1128 | 1120 |
| 71 | 1091 | 1080 | 1091 | 1091 | 1095 | 1108 | 1128 | 1121 |
| 72 | 1091 | 1080 | 1092 | 1091 | 1095 | 1107 | 1128 | 1121 |
| 73 | 1091 | 1080 | 1093 | 1092 | 1096 | 1108 | 1128 | 1122 |
| 74 | 1091 | 1080 | 1093 | 1091 | 1095 | 1107 | 1129 | 1121 |
| 75 | 1091 | 1080 | 1092 | 1091 | 1095 | 1107 | 1128 | 1120 |
| 76 | 1089 | 1080 | 1091 | 1091 | 1095 | 1108 | 1127 | 1120 |
| 77 | 1091 | 1081 | 1092 | 1091 | 1095 | 1108 | 1129 | 1120 |
| 78 | 1091 | 1080 | 1092 | 1091 | 1094 | 1108 | 1129 | 1120 |
| 79 | 1096 | 1086 | 1092 | 1092 | 1106 | 1115 | 1133 | 1124 |
| 80 | 1098 | 1085 | 1092 | 1088 | 1101 | 1112 | 1130 | 1122 |
| 81 | 1096 | 1086 | 1092 | 1089 | 1101 | 1110 | 1130 | 1120 |
| 82 | 1096 | 1086 | 1092 | 1088 | 1102 | 1112 | 1130 | 1121 |
| 83 | 1096 | 1086 | 1091 | 1088 | 1101 | 1112 | 1130 | 1121 |
| 84 | 1098 | 1086 | 1091 | 1088 | 1103 | 1110 | 1129 | 1121 |
| 85 | 1096 | 1086 | 1092 | 1088 | 1102 | 1110 | 1129 | 1121 |
| 86 | 1098 | 1087 | 1092 | 1089 | 1102 | 1112 | 1130 | 1121 |
| 87 | 1099 | 1088 | 1092 | 1091 | 1103 | 1114 | 1133 | 1123 |
| 88 | 1101 | 1091 | 1094 | 1093 | 1106 | 1116 | 1134 | 1126 |
| 89 | 1103 | 1093 | 1098 | 1094 | 1109 | 1119 | 1137 | 1127 |
| 90 | 1105 | 1092 | 1098 | 1094 | 1109 | 1117 | 1136 | 1128 |
| 91 | 1103 | 1089 | 1096 | 1093 | 1108 | 1115 | 1135 | 1126 |
| 92 | 1101 | 1087 | 1094 | 1091 | 1106 | 1114 | 1133 | 1122 |
| 93 | 1098 | 1085 | 1091 | 1088 | 1102 | 1112 | 1130 | 1121 |
| 94 | 1095 | 1082 | 1089 | 1085 | 1099 | 1108 | 1127 | 1117 |
| 95 | 1093 | 1086 | 1088 | 1089 | 1099 | 1113 | 1127 | 1122 |
| 96 | 1095 | 1082 | 1092 | 1089 | 1098 | 1109 | 1129 | 1122 |
| 97 | 1091 | 1083 | 1091 | 1091 | 1095 | 1109 | 1127 | 1121 |
| 98 | 1091 | 1083 | 1092 | 1092 | 1095 | 1110 | 1127 | 1121 |
| 99 | 1092 | 1082 | 1092 | 1092 | 1095 | 1110 | 1127 | 1121 |
| 100 | 1091 | 1081 | 1092 | 1092 | 1094 | 1109 | 1128 | 1121 |
| 101 | 1091 | 1082 | 1092 | 1091 | 1094 | 1109 | 1127 | 1122 |
| 102 | 1089 | 1081 | 1092 | 1091 | 1094 | 1109 | 1127 | 1122 |
| 103 | 1091 | 1082 | 1092 | 1091 | 1095 | 1109 | 1127 | 1121 |
| 104 | 1091 | 1081 | 1089 | 1091 | 1095 | 1109 | 1128 | 1122 |
| 105 | 1091 | 1081 | 1091 | 1089 | 1095 | 1109 | 1127 | 1121 |
| 106 | 1089 | 1081 | 1091 | 1089 | 1095 | 1108 | 1127 | 1121 |
| 107 | 1089 | 1081 | 1092 | 1091 | 1094 | 1108 | 1126 | 1121 |
| 108 | 1091 | 1080 | 1091 | 1091 | 1094 | 1109 | 1126 | 1120 |
| 109 | 1088 | 1081 | 1091 | 1091 | 1094 | 1108 | 1126 | 1119 |

TABLE 4
aggregated resistance values (ohms) across channels 1-16

| Time | | | | | weight |
|---|---|---|---|---|---|
| elapse | Total | Bottom | Middle | Top | average |
| 1 | 1 | 1 | 1 | 1 | 1 |
| 2 | 1.004958 | 1.014874 | 1 | 1 | 1.008924145 |
| 3 | 0.99793 | 1.004958 | 0.994417 | 0.994417 | 1.000741326 |
| 4 | 0.990486 | 1.004958 | 0.98325 | 0.98325 | 0.996274547 |
| 5 | 1.005375 | 1.004958 | 1.005583 | 1.005583 | 1.005208104 |
| 6 | 1.00207 | 0.995042 | 1.005583 | 1.005583 | 0.999258674 |
| 7 | 0.990486 | 1.004958 | 0.98325 | 0.98325 | 0.996274547 |
| 8 | 0.992555 | 1 | 0.988833 | 0.988833 | 0.995533222 |
| 9 | 1.001653 | 1.004958 | 1 | 1 | 1.002974715 |
| 10 | 1.009097 | 1.004958 | 1.011167 | 1.011167 | 1.007441493 |
| 11 | 0.990903 | 0.995042 | 0.988833 | 0.988833 | 0.992558507 |
| 12 | 0.992555 | 1 | 0.988833 | 0.988833 | 0.995533222 |
| 13 | 0.984292 | 0.975211 | 0.988833 | 0.988833 | 0.980659647 |
| 14 | 0.98925 | 0.990084 | 0.988833 | 0.988833 | 0.989583792 |
| 15 | 1.004958 | 1.014874 | 1 | 1 | 1.008924145 |
| 16 | 1.003722 | 1 | 1.005583 | 1.005583 | 1.002233389 |
| 17 | 1.031833 | 1.039663 | 1.027917 | 1.027917 | 1.034964665 |
| 18 | 1.060777 | 1.059494 | 1.061418 | 1.061418 | 1.06026386 |
| 19 | 1.05168 | 1.054536 | 1.050251 | 1.050251 | 1.052822367 |
| 20 | 1.042165 | 1.059494 | 1.033501 | 1.033501 | 1.049096914 |
| 21 | 1.06243 | 1.064452 | 1.061418 | 1.061418 | 1.063238575 |
| 22 | 1.067604 | 1.06941 | 1.067002 | 1.067002 | 1.068446679 |
| 23 | 1.075249 | 1.06941 | 1.078169 | 1.078169 | 1.072913457 |
| 24 | 1.070291 | 1.054536 | 1.078169 | 1.078169 | 1.063989313 |
| 25 | 1.060777 | 1.059494 | 1.061418 | 1.061418 | 1.06026386 |
| 26 | 1.04754 | 1.064452 | 1.039084 | 1.039084 | 1.054305018 |
| 27 | 1.064499 | 1.059494 | 1.067002 | 1.067002 | 1.062497249 |
| 28 | 1.076902 | 1.074368 | 1.078169 | 1.078169 | 1.075888172 |
| 29 | 1.065735 | 1.074368 | 1.061418 | 1.061418 | 1.069188005 |
| 30 | 1.064499 | 1.059494 | 1.067002 | 1.067002 | 1.062497249 |
| 31 | 1.068222 | 1.059494 | 1.072585 | 1.072585 | 1.064730638 |
| 32 | 1.06036 | 1.06941 | 1.055835 | 1.055835 | 1.063979901 |
| 33 | 1.05168 | 1.054536 | 1.050251 | 1.050251 | 1.052822367 |
| 34 | 1.051263 | 1.064452 | 1.044668 | 1.044668 | 1.056538407 |
| 35 | 1.03018 | 1.034705 | 1.027917 | 1.027917 | 1.03198995 |
| 36 | 1.037207 | 1.044621 | 1.033501 | 1.033501 | 1.040172769 |
| 37 | 1.04093 | 1.044621 | 1.039084 | 1.039084 | 1.042406158 |
| 38 | 1.031833 | 1.039663 | 1.027917 | 1.027917 | 1.034964665 |
| 39 | 1.035555 | 1.039663 | 1.033501 | 1.033501 | 1.037198054 |
| 40 | 1.033485 | 1.044621 | 1.027917 | 1.027917 | 1.03793938 |
| 41 | 1.042165 | 1.059494 | 1.033501 | 1.033501 | 1.049096914 |
| 42 | 1.037207 | 1.044621 | 1.033501 | 1.033501 | 1.040172769 |
| 43 | 1.035138 | 1.049579 | 1.027917 | 1.027917 | 1.040914095 |
| 44 | 1.040513 | 1.054536 | 1.033501 | 1.033501 | 1.046122199 |
| 45 | 1.029763 | 1.044621 | 1.022334 | 1.022334 | 1.035705991 |
| 46 | 1.029763 | 1.044621 | 1.022334 | 1.022334 | 1.035705991 |
| 47 | 1.03886 | 1.049579 | 1.033501 | 1.033501 | 1.043147484 |
| 48 | 1.082678 | 1.114031 | 1.067002 | 1.067002 | 1.095219113 |
| 49 | 1.169511 | 1.173525 | 1.167504 | 1.167504 | 1.171116697 |
| 50 | 1.176956 | 1.173525 | 1.178671 | 1.178671 | 1.175583476 |
| 51 | 1.176122 | 1.193356 | 1.167504 | 1.167504 | 1.183015557 |
| 52 | 1.188941 | 1.198314 | 1.184255 | 1.184255 | 1.192690439 |
| 53 | 1.185219 | 1.198314 | 1.178671 | 1.178671 | 1.19045705 |
| 54 | 1.201344 | 1.213188 | 1.195422 | 1.195422 | 1.206081363 |
| 55 | 1.186871 | 1.203272 | 1.178671 | 1.178671 | 1.193431765 |
| 56 | 1.185219 | 1.198314 | 1.178671 | 1.178671 | 1.19045705 |
| 57 | 1.194316 | 1.203272 | 1.189838 | 1.189838 | 1.197898544 |
| 58 | 1.200108 | 1.198314 | 1.201005 | 1.201005 | 1.199390607 |
| 59 | 1.213329 | 1.237977 | 1.201005 | 1.201005 | 1.223188326 |
| 60 | 1.201344 | 1.213188 | 1.195422 | 1.195422 | 1.206081363 |
| 61 | 1.186454 | 1.213188 | 1.173088 | 1.173088 | 1.197147806 |
| 62 | 1.212094 | 1.223104 | 1.206588 | 1.206588 | 1.216497571 |
| 63 | 1.171982 | 1.203272 | 1.156337 | 1.156337 | 1.184498209 |
| 64 | 1.083095 | 1.104115 | 1.072585 | 1.072585 | 1.091503073 |
| 65 | 1.050845 | 1.074368 | 1.039084 | 1.039084 | 1.060254448 |
| 66 | 1.032651 | 1.064452 | 1.01675 | 1.01675 | 1.045371462 |
| 67 | 1.040513 | 1.054536 | 1.033501 | 1.033501 | 1.046122199 |
| 68 | 1.029763 | 1.044621 | 1.022334 | 1.022334 | 1.035705991 |
| 69 | 1.018179 | 1.054536 | 1 | 1 | 1.032721864 |
| 70 | 1.030998 | 1.059494 | 1.01675 | 1.01675 | 1.042396747 |
| 71 | 1.029346 | 1.054536 | 1.01675 | 1.01675 | 1.039422032 |
| 72 | 1.03679 | 1.054536 | 1.027917 | 1.027917 | 1.04388881 |
| 73 | 1.038443 | 1.059494 | 1.027917 | 1.027917 | 1.046863525 |
| 74 | 1.035138 | 1.049579 | 1.027917 | 1.027917 | 1.040914095 |
| 75 | 1.042165 | 1.059494 | 1.033501 | 1.033501 | 1.049096914 |
| 76 | 1.020249 | 1.049579 | 1.005583 | 1.005583 | 1.031980538 |
| 77 | 1.045888 | 1.059494 | 1.039084 | 1.039084 | 1.051330303 |
| 78 | 1.031415 | 1.049579 | 1.022334 | 1.022334 | 1.038680706 |
| 79 | 1.15958 | 1.188399 | 1.14517 | 1.14517 | 1.171107285 |
| 80 | 1.248451 | 1.332176 | 1.206588 | 1.206588 | 1.281941299 |
| 81 | 1.220341 | 1.292514 | 1.184255 | 1.184255 | 1.249210023 |
| 82 | 1.229438 | 1.297471 | 1.195422 | 1.195422 | 1.256651516 |
| 83 | 1.223646 | 1.302429 | 1.184255 | 1.184255 | 1.255159453 |
| 84 | 1.236883 | 1.297471 | 1.206588 | 1.206588 | 1.261118295 |
| 85 | 1.231925 | 1.282598 | 1.206588 | 1.206588 | 1.25219415 |
| 86 | 1.247633 | 1.307387 | 1.217755 | 1.217755 | 1.271534503 |
| 87 | 1.27616 | 1.337134 | 1.245673 | 1.245673 | 1.300549738 |
| 88 | 1.340242 | 1.406544 | 1.307091 | 1.307091 | 1.366763028 |
| 89 | 1.403506 | 1.451165 | 1.379676 | 1.379676 | 1.422569521 |
| 90 | 1.399366 | 1.461081 | 1.368509 | 1.368509 | 1.424052173 |
| 91 | 1.351409 | 1.406544 | 1.323841 | 1.323841 | 1.373463195 |
| 82 | 1.292702 | 1.342092 | 1.268007 | 1.268007 | 1.31245801 |
| 93 | 1.203398 | 1.252851 | 1.178671 | 1.178671 | 1.223178914 |
| 94 | 1.138898 | 1.193356 | 1.111669 | 1.111669 | 1.160681665 |
| 95 | 1.193868 | 1.302429 | 1.139587 | 1.139587 | 1.23729234 |
| 96 | 1.126079 | 1.188399 | 1.094919 | 1.094919 | 1.151006783 |
| 97 | 1.095899 | 1.153694 | 1.067002 | 1.067002 | 1.119016833 |
| 98 | 1.101691 | 1.148736 | 1.078169 | 1.078169 | 1.120508896 |
| 99 | 1.090941 | 1.13882 | 1.067002 | 1.067002 | 1.110092688 |
| 100 | 1.078539 | 1.123946 | 1.055835 | 1.055835 | 1.096701765 |
| 101 | 1.075233 | 1.114031 | 1.055835 | 1.055835 | 1.090752335 |
| 102 | 1.059109 | 1.099157 | 1.039084 | 1.039084 | 1.075128023 |
| 103 | 1.082261 | 1.123946 | 1.061418 | 1.061418 | 1.098935154 |
| 104 | 1.065719 | 1.118989 | 1.039084 | 1.039084 | 1.087026882 |
| 105 | 1.066553 | 1.099157 | 1.050251 | 1.050251 | 1.079594801 |
| 106 | 1.050011 | 1.094199 | 1.027917 | 1.027917 | 1.067686529 |
| 107 | 1.052081 | 1.089241 | 1.033501 | 1.033501 | 1.066945204 |
| 108 | 1.042984 | 1.084284 | 1.022334 | 1.022334 | 1.05950371 |
| 109 | 1.045053 | 1.079326 | 1.027917 | 1.027917 | 1.058762385 |

1-7. (canceled)

8. An apparatus comprising at least one sensor to detect the position and/or orientation of a body portion of a subject, the at least one sensor comprising:
a substrate layer;
a resistive ink layer positioned above the substrate layer; and
a dielectric layer positioned above the substrate layer, wherein the substrate layer, resistive ink layer, and dielectric layer are configured to collectively stretch in multi-dimensional movements while adhered to the body portion of the subject.

9. The apparatus of claim 8, wherein the substrate layer, resistive ink layer, and dielectric layer are configured to displace when stretching in a manner similar to the human skin.

10. The apparatus of claim 8, wherein the multi-dimensional movements comprises horizontal, vertical, and diagonal movements.

11. The apparatus of claim 8, further comprising an adhesive backing layer positioned below the substrate layer for placing the at least one sensor on the body portion of the subject.

12. The apparatus of claim 8, further comprising a conductive ink layer positioned above the substrate.

13. The apparatus of claim 8, wherein at least one of the substrate layer, resistive ink layer, and dielectric layer is of sufficient strength to avoid tearing during stretching in the multidimensional movements.

14. The apparatus of claim 8, further comprising a computer processor configured to receive a plurality of resistance values from the at least one sensor and determine the body portion position and/or orientation based on the plurality of resistance values.
